(19) United States
(12) Patent Application Publication
(10) Pub. No.: US 2023/0201341 A1
(43) Pub. Date: Jun. 29, 2023

(54) TLR3 AND TLR9 AGONISTS AS VACCINE ADJUVANTS FOR ANTI-CANCER DNA VACCINES

(71) Applicant: WISCONSIN ALUMNI RESEARCH FOUNDATION, Madison, WI (US)

(72) Inventors: Douglas McNeel, Madison, WI (US); Donghwan Jeon, Madison, WI (US)

(21) Appl. No.: 18/053,659

(22) Filed: Nov. 8, 2022

Related U.S. Application Data
(60) Provisional application No. 63/276,933, filed on Nov. 8, 2021.

(51) Int. Cl. A61K 39/39; A61K 39/00; A61P 35/00

(52) U.S. Cl. CPC A61K 39/39; A61K 39/001193; A61P 35/00; A61K 2039/53; A61K 2039/55561; A61K 2039/55511

(57) ABSTRACT

The present invention provides the combination of TLR3 agonist and TLR9 agonist can be used to increase the anti-tumor immune response to vaccines, particularly nucleic acid vaccines. Methods of treating cancer and inducing anti-tumor immune responses by treating with a TLR3 agonist and a TLR9 agonist in combination with administration of an anti-tumor vaccine are provided. The TLR3 agonist, TLR9 agonist and the vaccine may be co-administered in a unitary composition or the agonists may be administered in advance of the vaccine.

### CROSS-REFERENCE TO RELATED APPLICATIONS

This application claims priority to U.S. Provisional Application No. 63/276,933 filed on Nov. 8, 2021, the content of which is incorporated by reference herein in its entirety.

### STATEMENT REGARDING FEDERALLY SPONSORED RESEARCH

This invention was made with government support under CA219154 awarded by the National Institutes of Health. The government has certain rights in the invention.

### SEQUENCE LISTING

A Sequence Listing accompanies this application and is submitted as an XML file of the sequence listing named “960296.04335 Sequence Listing.xml” which is 51,215 bytes in size and was created on Oct. 19, 2022, 2022. The sequence listing is electronically submitted via Patent Center with the application and is incorporated herein by reference in its entirety.

### BACKGROUND OF THE INVENTION

The field of the invention concerns improving the efficacy of immune responses to vaccines, particularly cancer vaccines and specifically DNA vaccines. The invention also relates to new treatment for cancer by enhancing the immune response against cancer cells in combination with a vaccine.

T-cell checkpoint receptors, such as cytotoxic T-lymphocyte-associated protein 4 (CTLA-4), programmed death 1 (PD-1), lymphocyte activation gene 3 (LAG-3), and T-cell immunoglobulin and mucin-domain containing 3 (TIM-3), were first discovered on T cells and associated with reduced functional phenotype of T-cells. This led to these receptors generally being considered as markers of T-cell exhaustion. However, the expression of these T-cell checkpoint receptors occurs normally when T cells are activated, and it is binding of the ligands to these receptors that leads to reduced effector function of T cells. Moreover, the timing and expression of these receptors can be affected by events during T-cell activation. For example, T cells activated by a high affinity epitope have increased PD-1 expression which can persist over time, while T cells stimulated with a lower affinity antigen became activated with lower PD-1 expression. This altered expression of PD-1, in particular, has consequences for anti-tumor immunity; lower expression of PD-1, or blockade of PD-1/PD-L1, has been demonstrated to lead to greater anti-tumor efficacy in murine models in which CD8 T cells were activated with vaccination. These findings suggest that efforts to lower or block the expression of multiple checkpoint receptors could have profound effects on the efficacy of activated CD8 T cells used as anti-tumor therapies.

Certain pathogens can induce robust immune responses, which serves as the basis for using them as either delivery vehicles for vaccine antigens, or as vaccine adjuvants. The recent adenovirus vaccine developed by Janssen for SARS-CoV-2 serves as an example. This rapid innate immune response is related to the activation of pattern recognition receptors (PRRs), such as toll-like receptors (TLRs).

TLRs are transmembrane receptor proteins which activate the innate immune system by sensing diverse molecules from microorganisms, called Pathogen Associated Molecular Patterns (PAMPs), or Danger Associated Molecular Patterns (DAMPs). Ten different TLRs have been identified in humans and mice (TLR1-10 for humans, TLR1-9 and 13 for mice). They are expressed on macrophages and dendritic cells, as well as non-immune cells, such as epithelial cells or fibroblasts. Each TLR recognizes a different type of biomolecule, and stimulation of these receptors leads to the activation of innate and adaptive immune responses. Like other adjuvants which augment immune responses, chemical agonists for TLRs have been widely explored as adjuvants for traditional vaccines. Many investigators have also evaluated TLR agonists as adjuvants for anti-cancer vaccines. However, some of these approaches have had limited success in increasing the immunogenicity in vivo and thus there is a need for other methods to increase the anti-tumor response.

Prostate cancer is a significant health risk for men over 50, with about 200,000 newly diagnosed cases each year in the United States (Jemal A. et al., Cancer Statistics, 2005 (2005) CA Cancer J Clin, 55:10-30). It is the most common tumor diagnosed among men and the second leading cause of male cancer-related death in the United States (Jemal et al., Cancer Statistics, 2003 (2003) CA Cancer J Clin, 53:5-26). Despite advances in screening and early detection, approximately 30% of patients undergoing definitive prostatectomy or ablative radiation therapy will have recurrent disease at 10 years (Oefelein et al., 1997, J Urol, 158:1460-1465). While immune checkpoint blockades have gained traction in treating many cancers, prostate cancer has been notoriously unaffected by this type of immunotherapeutic agent.

Therefore, there is a need to increase the immunogenicity of DNA vaccines by regulating the immune response and to increase the anti-tumor activity of the immune cells. Specifically, there is a need to overcome the low immunogenicity of DNA vaccines toward cancers, including methods for increasing the CD8+ T cell response to tumor antigens.

### SUMMARY OF THE INVENTION

The present invention overcomes the aforementioned drawbacks by providing improved responses to vaccines by administering TLR3 agonist or TLR9 agonists in combination with an immunotherapeutic agent, for example, a vaccine for inducing an anti-tumor immune response.

In a first aspect, the disclosure provides a method of eliciting an anti-tumor response in a subject in need thereof, the method comprising administering at least one TLR3 agonist, at least one TLR9 agonist and an anti-tumor vaccine to the subject in an effective amount to elicit an anti-tumor response in said subject.

In another aspect, the disclosure provides a method of increasing the immune response to a cancer vaccine, the method comprising administering at least one TLR3 agonist and at least one TLR9 agonist and at least one vaccine directed to at least one tumor antigen in a subject, wherein the immune response to the tumor antigen is increased relative to a subject treated with the vaccine alone.

In a further aspect, the disclosure provides a kit for eliciting an anti-tumor response, the kit comprising at least one TLR3 agonist and at least one TLR9 agonist; and at least one cancer vaccine.

In a further embodiment, the disclosure provides a composition for eliciting an anti-tumor response, the composition comprising at least one TLR3 agonist, at least one TLR9 agonist and at least one vaccine.

The foregoing and other aspects and advantages of the invention will appear in the following description. In the description, reference is made to the accompanying drawings which form a part hereof, and in which there is shown by way of illustration preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Combination of TLR agonists at the time of T-cell activation in vitro affects expression of T-cell checkpoint receptors: Splenocytes were prepared from the spleens of OT-1 mice and stimulated in vitro with the high-affinity SIINFEKL (OVA) peptide in the presence or absence of TLR agonists [TLR 1/2 (Pam3CSK4), TLR 3 (Poly I:C), TLR 4 (MPLAs), TLR 5 (FLA-ST), TLR 2/6 (FSL-1), TLR 7 (Gardiquimod), TLR 7/8 (R848), TLR 9 (ODN1826), or TLR 13 (ORN Sal 9)] or their pairwise combinations. The mean fluorescence intensity (MFI) of 4-1BB and T-cell checkpoint receptor expression on CD8+ T cells were determined by flow cytometry, collected daily for 4 days, and computed as Area Under the Curve (AUC) using the trapezoid rule. (A) Calculation of AUC ratio to compare AUC of each receptor expression for each pairwise combination with that obtained following OVA stimulation alone, without TLR activation. (B) Heat-map demonstrating AUC ratio of each pairwise combination for 4-1BB and multiple T-cell checkpoint receptors. (C) Representative MFI plots showing 4-1BB, PD-1, LAG-3, and CD160 expression with the combinations of TLR1/2, TLR3, and TLR9 agonists. Asterisks represent significant differences (p<0.05). Heat-maps of AUC ratios shown in Figure B were generated by R 3.3.1.

FIG. 2. Combination of TLR agonists elicits greater antitumor immunity in vivo. Ovalbumin-expressing E.G7 cells were implanted in C57BL/6 mice and permitted to grow until tumors were palpable (10 days). OT-1 splenocytes were then adoptively transferred and mice were immunized subcutaneously the following day with SIINFEKL (OVA) peptide alone, or in combination with TLR agonists, the day following immunization. (A) Shown are the tumor growth curves (median+standard error, n=6 to 7 animals per group). (B) Tumors obtained at necropsy were evaluated for the percentage of infiltrating tetramer⁺ CD8⁺ T cells among CD45⁺ cells. Panel (C): Tumor-infiltrating tetramer⁺ CD8⁺ T cells were evaluated for 4-1BB and PD-1 expression by flow cytometry. Asterisks represent significant differences (p<0.05). Results are from one experiment and are representative of two independent experiments.

FIG. 3. Anti-tumor effects of TLR combinations not strictly dependent on IL12. (A) Purified DCs were stimulated in the presence of TLR1/2 agonist (Pam3CSK4), TLR3 agonist (HMW PolyI:C), TLR9 agonist (ODN 1826), pairwise combinations of these TLR agonists, or media only (no TLRs) for 24 hours. Culture supernatant was then evaluated for the presence of IL12 p70 by ELISA. (B) Ovalbumin-expressing E.G7 cells were implanted in IL12p40 deficient mice and permitted to grow until tumors were palpable (11 days). OT-1 CD8+ T cells were then adoptively transferred, and mice were immunized subcutaneously the following day with SIINFEKL (OVA) peptide alone, or in combination with TLR agonists. Shown are the tumor growth curves (median+standard error, n=5 animals per group). Asterisks show significant differences (p<0.05).

FIG. 4. CD8⁺ T cells activated in the presence of TLR1/2, TLR3, TLR9 agonists exhibit distinct gene expression profiles with differences in effector and memory function. OT-1 splenocytes were activated for 72 hours in the presence of SIINFEKL peptide (OVA) alone, or with TLR1/2, TLR3, and/or TLR9 agonists. CD8+OT-1 T cells were then purified and evaluated for gene-expression changes by RNA-seq. All samples were evaluated in six replicates from a single experiment. (A) Representative color codes for each treatment groups for subsequent analysis. (B) Principle-Component Analysis plot for each treatment group. (C) Heat-map of gene expression changes for specific genes associated with CD8+T cell differentiation and function for CD8+T cells stimulated with OVA in the presence of TLR agonists versus CD8+T cells stimulated with OVA alone (red=increased expression, blue=reduced expression, with fold-change indicated by the numbers). (D) Table showing enriched gene sets from pre-ranked GSEA between each treatment group. Gene sets identified in at least 5 of the pairwise comparisons were selected. Numbers indicate fold change and (p value) for each of the 9 pairwise comparisons.

FIG. 5. Changes in CD8+T cell expression of PD-1 following TLR stimulation depends on both IL-12 and type-1 interferons. (A) Purified DCs or B cells were stimulated in the presence of TLR1/2 agonist (Pam3CSK4), TLR3 agonist (HMW PolyI:C), TLR9 agonist (ODN 1826), or media only (Control) for 24 hours. Culture supernatant was then evaluated for the presence of IFNβ by ELISA. (B) OT-1 splenocytes were stimulated in vitro with the high-affinity SIINFEKL peptide in the presence of specific TLR agonists, anti-IFNAR1 and/or anti-IL12Rβ2. The mean fluorescence intensity (MFI) of PD-1 expression on CD8⁺ T cells was assessed after 3 days, by flow cytometry. (C) OT-1 splenocytes were stimulated in vitro with the high-affinity SIINFEKL peptide in the presence of recombinant IL12 and/or IFNβ. The mean fluorescence intensity (MFI) of 4-1BB and PD-1 expression on CD8+ T cells were assessed after 4 days, by flow cytometry. Asterisks demonstrate p<0.05.

FIG. 6. Combinations of TLR agonists improve the anti-tumor efficacy of a DNA vaccine in a prostate cancer tumor model. C57BL/6 mice were implanted subcutaneously with TRAMP-C1 tumor cells. Mice were immunized intradermally weekly with control vector (pTVG4) or DNA encoding AR ligand-binding domain (pTVG-AR) and delivered alone or co-delivered with TLR1/2, TLR3 and/or TLR9 agonists. (A) shown are the growth curves for each group (n=6 animals per group). (B) Survival plots using the time to death or when tumors reached 2 cm3 in size, whichever occurred first. (C) Tumors obtained at necropsy were evaluated for the percentage of infiltrating CD45⁺ cells, CD8⁺ T cells among CD45⁺ cells, and T_{reg} cells (CD25⁺Foxp3⁺) among CD4⁺CD45⁺ cells. (D) Tumor-infiltrating CD8+ T cells were evaluated for 4-1BB and PD-1 expression by flow cytometry. In all panels, asterisks demonstrate p<0.05.

FIG. 7. Combination of TLR agonists at the time of T-cell activation in vitro affects expression of T-cell checkpoint receptors. Splenocytes were prepared from the spleens of OT-1 mice and stimulated in vitro with the high-affinity SIINFEKL peptide in the presence or absence of specific TLR agonists [TLR 1/2 (Pam3CSK4), TLR 3 (Poly I:C), or TLR 9 (ODN1826). The mean fluorescence intensity (MFI) of the expression of 41BB, PD-1, LAG-3, CD160, CD244.2, CTLA4, TIM-3, TIGIT, and VISTA on CD8+ T cells were collected daily for 4 days. Representative MFI plots are shown for (A) TLR1/2+TLR3, (B) TLR1/2+TLR9, and (C) TLR3+TLR9 combinations. (D) Splenocytes prepared from the spleens of OT-1 mice were stimulated in vitro with the high-affinity SIINFEKL peptide in the presence or absence of TLR3 or TLR9 agonists in different concentrations (TLR3: 0, 1, 2.5, 10 μg/ml, TLR9: 0, 0.5 1.25, 5 μM). The mean fluorescence intensity (MFI) of the expression of 41BB, PD-1, CD160, LAG-3 TIM-3, and CTLA-4 on CD8+ T cells were collected after 72 hours using flow cytometry. Shown are heat-maps of the fold-change in MFI relative to treatment with OVA alone. Asterisks demonstrate p<0.05.

FIG. 8. Combination of TLR agonists elicits greater antitumor immunity in vivo. Ovalbumin-expressing E.G7 cells were implanted in C57BL/6 mice and permitted to grow until tumors were palpable (10 days). OT-1 splenocytes were then adoptively transferred and mice were immunized subcutaneously the following day with SIINFEKL (OVA) peptide alone, or in combination with TLR agonists, the day following immunization. Shown are the tumor growth curves for individual mice (A), and a Kaplan-Meir survival plot (B), using the time to death or when tumors reached 2 cm³ in size, whichever occurred first. Asterisks indicate p<0.05 as assessed by log-rank test.

FIG. 9. DC secrete IL12 following stimulation with TLR agonist combinations, but anti-tumor effects of TLR combinations are not strictly dependent on IL12. Purified DCs were stimulated in the presence of TLR agonists [TLR 1/2 (Pam3CSK4), TLR 3 (Poly I:C), TLR 4 (MPLAs), TLR 5 (FLA-ST), TLR 2/6 (FSL-1), TLR 7 (Gardiquimod), TLR 7/8 (R848), TLR 9 (ODN1826))] or their pairwise combinations for 24 hours. Culture supernatant was then evaluated for the presence of IL12 p70 by ELISA (A). Ovalbumin-expressing E.G7 cells were implanted in IL12p40 deficient mice and permitted to grow until tumors were palpable (11 days). OT-1 CD8+ T cells were then adoptively transferred, and mice were immunized subcutaneously the following day with SIINFEKL (OVA) peptide alone, or in combination with TLR agonists. The tumor growth curves for individual mice are shown in (B), and Kaplan-Meir survival plots (C) were generated using the time to death or when tumors reached 2 cm³ in size, whichever occurred first. Asterisks indicate p<0.05 as assessed by log-rank test.

FIG. 10. Changes in CD8+T cell expression of specific checkpoint receptors following TLR stimulation depends on both IL-12 and type-1 interferons. (A) OT-1 splenocytes were stimulated in vitro with the high-affinity SIINFEKL peptide in the presence of TLR1/2 agonist (Pam3CSK4), TLR3 agonist (HMW PolyI:C), TLR9 agonist (ODN 1826), anti-IFNAR1 and/or anti-IL12Rβ2. The mean fluorescence intensity (MFI) of 4-1BB, CD160 and LAG3 expression on CD8⁺ T cells was assessed after 3 days, by flow cytometry. (B) OT-1 splenocytes were stimulated in vitro with the high-affinity SIINFEKL peptide in the presence of recombinant IL12 and/or IFNβ. The mean fluorescence intensity (MFI) of CD160 and LAG-3 expression on CD8⁺ T cells was assessed after 4 days, by flow cytometry.

FIG. 11. Combinations of TLR agonists improve the anti-tumor efficacy of a DNA vaccine in a prostate cancer tumor model. C57BL/6 mice were implanted subcutaneously with TRAMP-C1 tumor cells. Mice were immunized intradermally weekly with control vector (pTVG4) or DNA encoding AR ligand-binding domain (pTVG-AR) and delivered alone or co-delivered with TLR1/2, TLR3 and/or TLR9 agonists. Shown are the tumor growth curves for individual mice in each group (n=6 animals per group).

### DETAILED DESCRIPTION OF THE INVENTION

The present invention has been described in terms of one or more preferred embodiments, and it should be appreciated that many equivalents, alternatives, variations, and modifications other than those expressly stated are also possible and within the scope of the invention.

The present disclosure is directed to the surprising finding that agonists of specific TLRs, specifically TLR3 and TLR9, are the most effective at activating T cells and inducing anti-tumor activity, specifically the anti-tumor response, of activated immune cells (e.g., CD8+T cells).

The specific combinations of TLR agonists when used at the time of T-cell activation with vaccines suppresses the expression of multiple T-cell checkpoint receptors, affects CD8+T cell function, and elicits greater antitumor activity. The inventors have demonstrated that specific T-cell checkpoint receptor expression, notably PD-1, on CD8+T cells following stimulation with the antigen alone or with combinations of different TLR agonists is diminished. The inventors demonstrate that administration of combinations of particular TLR agonists down-regulated the expression of certain T cell checkpoint receptors maintained an activated phenotype and are able to elicit an anti-tumor activity in murine tumor models when used with peptide or DNA vaccines.

In one embodiment, the disclosure provides a method of eliciting an anti-tumor response in a subject in need thereof, the method comprising administering at least one TLR3 agonist and at least one TLR9 agonist and an anti-tumor vaccine to the subject in an effective amount to elicit an anti-tumor response in said subject. This increased anti-tumor response reduces and inhibits tumor growth, and, in some cases, tumor ablation, in an in vivo cancer model. The inventors have found that when a vaccine is administered in combination with at least one TLR3 agonist and at least one TLR9 agonist, antigen-specific immune cells are activated and the immune response to the tumor antigen is enhanced. This result is specific to TLR3 agonist and TLR9 agonist in combination and is not applicable to all or other TLR agonists. The survival of mice was significantly increased in the mice that were treated with both TLR3 and TLR9 agonist with the DNA vaccine. Further, there is an increase in IL-12 and interferon by the administration of the combination composition comprised herein.

Immunization using an anti-tumor DNA vaccine increases expression in tumor cells of PD-L1, which is associated with the development of tumor antigen-specific IFNγ secretion, in both animals and human patients. Without being bound by any theory, it is thought that, at least in some instances, increased PD-L1 expression inactivates activated and trafficked immune cells, and down-regulates cell-mediated immune responses. In other words, it is believed that when these high PD-1-expressing T cells reach tumor cells decorated with PD-L1, these T cells are more likely to be deactivated than T cells expressing lower levels of PD-1, such that the cytotoxic immune response against the tumor cells is decreased. Such T cells include T cells that are activated by an enhanced antigen (i.e. antigen that have been modified to increase their affinity for a TCR) as these T cells express higher levels of PD-1.

The anti-tumor response could be restored if vaccination was combined with PD-1/PD-L1 blockade. Id. However, anti-PD-1 treatment has had limited success in vivo for some cancers like prostate cancer. The inventors have shown that blocking the PD-1 pathway would beneficially improve immunogenicity of cancer vaccines and have also surprisingly found that TLR3 agonists and TLR9 agonists can reduce expression of PD-1 on activated immune cells and increase immune response to the target cancer antigen.

The inventors here show that TLR agonists TLR3 and TLR9 together unexpectedly improve vaccine efficacy by increasing the anti-tumor function of immune cells specific for the target cancer antigen that are activated by vaccination. The present disclosure provides methods for improving efficacy of vaccines, including cancer vaccines, and for improving anti-tumor responses, by administering TLR agonists, specifically TLR3 agonist and TLR9 agonists in combination with cancer vaccine. Data obtained in a mouse model system demonstrates that treating a tumor with both TLR3 agonist (e.g., HMW PolyI:C)) or TLR9 agonist (ODN 1826) improved the tumor growth suppression when administered with a DNA cancer vaccine in mice. Stimulation with TLR agonists led to an almost complete tumor growth suppression, significantly better than the effect seen in animals given the vaccine alone. Other TLR agonists do not have such an effect or anti-tumor responses as demonstrated in the examples below. Using mouse models, the inventors demonstrated that this combination results in greater anti-tumor efficacy when compared to the vaccine or agonists alone. Thus treatment with TLR agonists combined with strategies to increase tumor-specific CD8+T cells results in an increase in activated and unregulated tumor-specific T cells that are less susceptible to immune checkpoint mediated regulation, opening up the possibility for more effective cancer treatments.

The methods provided herein comprise administering at least one TLR3 agonist and at least one TLR9 agonist in combination with at least one cancer vaccine to a subject in order to improve the efficacy of the vaccine, improve, enhance and/or increase the immune response against a specific tumor antigen, and/or increase the anti-tumor response in a subject. In some embodiments, a combination of at least one TLR3 agonist and at least one TLR9 agonist are both administered at the same time to the subject.

In some embodiments, the disclosure provides a method of increasing the immune response to a tumor antigen, the method comprising administering at least one TLR3 agonist and at least one TLR9 agonist and at least cancer vaccine (e.g., DNA cancer vaccine) directed to the tumor antigen in a subject, wherein the immune response to the tumor antigen is increased. In some embodiments, the immune response is a cellular immune response, specifically a CD8+ T cell response.

An “effective treatment” refers to treatment producing a beneficial effect, e.g., amelioration of at least one symptom of a disease or cancer. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing one or more symptoms of the cancer or metastasis thereof. Such effective treatment may, e.g., reduce patient pain, reduce the size or number of cancer cells, reduce or prevent metastasis of a cancer cell, or may slow cancer or metastatic cell growth.

The terms “effective amount” or “therapeutically effective amount” refer to an amount sufficient to effect beneficial or desirable biological or clinical results. That result can be reducing, alleviating, inhibiting or preventing one or more symptoms of a disease or condition, reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. In some embodiments, the effective amount is an amount suitable to provide the desired effect, e.g., anti-antigen or anti-tumor response.

The terms “metastasis” or “secondary tumor” refer to cancer cells that have spread to a secondary site, e.g., outside of the original primary cancer site. Secondary sites include, but are not limited to, for example, the lymphatic system, skin, distant organs (e.g., liver, stomach, pancreas, brain, etc.) and the like and will differ depending on the site of the primary tumor.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a mammalian, for example, human subject. For example, a suitable subject includes a subject in need of cancer treatment, particularly prostate cancer treatment.

The term “cellular response” refers to an immune response that involves the activation of T cells within an organism against an antigen, in particular as described herein, a tumor antigen. The T cell response can be the T cell directly killing the cell having the tumor antigens on its surface.

The term “CD8+ T cell response” refers to the activation of CD8+ cells to kill cells expressing the antigen to which the T cell was activated. CD8+T cells may kill the cells through different mechanisms, including, the secretion of cytokines, primarily TNF-α and IFN-γ, which have anti-tumor effects, the production and release of cytotoxic granules, e.g., perforin, and granzymes, and/or destruction of infected cells is via Fas/FasL interactions.

In some embodiments, the methods increase the anti-tumor response. Such increase in the anti-tumor response may be demonstrated by an increased anti-tumor response in an animal model of the tumor as compared with the animal model without treatment or with administration of the T cell activating agent alone. For example, an increase anti-tumor response may be observed after the administration of the at least one TLR3 agonist and at least one TLR9 agonist in combination with a T cell activating agent, e.g., a vaccine (e.g., DNA vaccine) and demonstrated by a measurable increase in the anti-tumor response. The anti-tumor response may be demonstrated by a decrease in tumor size and volume within the animal or an increase in CD8+T cell activation as compared to treatment with the DNA vaccine alone or as compared to the untreated control.

The methods disclosed herein can include a conventional treatment regimen, which can be altered to include the steps of the methods described herein. The methods disclosed herein can include monitoring the patient to determine efficacy of treatment and further modifying the treatment in response to the monitoring.

TLR3 agonists and TLR9 agonists are known in the art and used herein include, but are not limited to, for example, TLR 3 agonists such as HMW PolyI:C. Poly(A:U), HILTONOL® (PolyICLC), Riboxxol, Rintatolimod, among others. Suitable TLR9 agonist include, but are not limited to, for example, ODN 1826), ODN2006, ODN2216, ODN2395, EMD 1201081, lefitolimod, PF-3512676 (aka CPG7909), ISS 1018, IMO-2055, CpG-28, SD-101, CMP-001, IMO-2125 (tilsotolimod), MGN1703, AZD1419, among others.

In one embodiment, the present disclosure provides a method of eliciting, enhancing or improving an anti-tumor response in a subject in need thereof. The method comprises administering at least one TLR3 and TLR9 agonist and an anti-tumor vaccine to the subject in an effective amount to elicit an anti-tumor response in said subject. In one example, the method provides delivery intradermally with vaccine, at the same time as vaccination. An alternative approach could be systemic delivery of the TLR3 and TLR9 agents at the time of vaccination. In some embodiments, the anti-tumor response is improved over the response to the vaccine alone. In some embodiments, the anti-tumor response is a cell-mediated immune response. In some embodiments, the activated T cells, e.g., CD8+T cells. Suitably, the subject is a human subject suffering from cancer, preferably prostate cancer.

An “improved immune-mediated anti-tumor response” means an increase in the ability of one or more immune cells to recognize tumor cells. In some instances, the improved immune-mediated anti-tumor response results in an increased ability of one or more immune cells to target/recognize and kill cancer cells (e.g., CD8+T cells). An improved immune-mediated anti-tumor response may be seen as a reduction in the number of cancer cells, inhibiting, retarding or slowing the growth of cancer cells, inhibiting, retarding or slowing the metastasis of cancer cells, increased infiltration of cytotoxic T cells into the tumor, or decreased inhibition of immune population within the tumor microenvironment.

The terms “cell-mediated immune response” or “cell-mediated immunity” refer to an immune response mediated by immune cells and does not involve antibodies (humoral immune response). Specifically, cell-mediated immune response includes antigen-specific cytotoxic T-lymphocytes (CD8+T cells) or activation of phagocytes. Phagocytes include white blood cells such as neutrophils, monocytes, macrophages, mast cells, and dendritic cells. In a preferred embodiment, the cell-mediated immune response is a cytotoxic T cell response or CD8+T cell response.

The disease, disorder or condition that can be treated by the methods of the present invention include cancer, more specifically prostate cancer.

The term “cancer vaccine” or “tumor vaccine” or “vaccine” refers to a biological preparation that contains antigen or immunogen that can elicit an anti-tumor immune response. A vaccine is administered to an individual in order to stimulate that individual's immune response to said antigen or immunogen.

In some embodiments, the antigen is a tumor antigen. In a preferred embodiment, the antigen is a tumor-associated antigen. A tumor antigen or tumor-associated antigen is an antigen that is preferentially expressed on the surface of a tumor cell and not expressed on normal, healthy cells.

The term “cancer” and “tumor” as used herein interchangeably and are meant to encompass any cancer, neoplastic and preneoplastic disease that is characterized by abnormal growth of cells. Cancer may, for example, be selected from the group consisting of colon carcinoma, breast cancer, pancreatic cancer, ovarian cancer, prostate cancer, head and neck cancer, lung cancer, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, fibrosarcoma, myxosarcoma, liposarcoma, chondrosarcoma, osteogenic sarcoma, chordoma, angiosarcoma, endotheliosarcoma, Ewing's sarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, endometrial cancer, lung carcinoma, small cell lung carcinoma, bladder carcinoma, epithelial carcinoma, glioblastomas, neuronomas, craniopharingiomas, schwannomas, glioma, astrocytoma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, childhood-null acute lymphoid leukemia (ALL), thymic ALL, B-cell ALL, acute megakaryocytic leukemia, Burkitt's lymphoma, and T cell leukemia, small and large non-small cell lung carcinoma, acute granulocytic leukemia, germ cell tumors, endometrial cancer, gastric cancer, hairy cell leukemia, thyroid cancer and other cancers known in the art. In a preferred embodiment, the cancer is prostate cancer.

In a particular embodiment, the subject may suffer from prostate cancer. The vaccine may be a nucleic acid vaccine, in particular, a DNA vaccine targeted to prostate cancer. Suitable vaccines are known in the art and include, for example, a recombinant DNA vaccine that encodes the androgen receptor or fragments thereof or a peptide vaccine comprising a polypeptide androgen receptor or fragments thereof. Suitable recombinant DNA vaccines are disclosed in U.S. Pat. Nos. 7,910,565, 8,513,210 and 8,962,590, entitled “Prostate cancer vaccine,” and U.S. Pat. No. 7,179,797 and U.S. application Ser. No. 11/615,778 entitled “Methods and compositions for treating prostate cancer using DNA vaccines,” which are incorporated by reference in their entireties. In some embodiments, the DNA vaccine comprises pTVG-AR (pTVG-AR or pTVG-ARLBD refer to the same vector and both designations are used interchangeably herein). pTVG-AR is a vector comprising the coding sequence for the ligand-binding domain of the human androgen receptor gene inserted into the pTVG4 vector to create the immunization vector pTVG-AR, as disclosed in U.S. Pat. No. 7,910,565, incorporated by reference in its entirety. According to another embodiment, the DNA vaccine comprises a polynucleotide operatively linked to a transcriptional regulatory element (e.g., a promoter such as a heterologous promoter) wherein the polynucleotide encodes a member selected from (i) a mammalian androgen receptor (e.g., a human androgen receptor), (ii) a fragment of the androgen receptor that comprises the ligand-binding domain, (iii) a fragment of the ligand-binding domain defined by SEQ ID NO:3(LLLFSIIPV); (iv) a fragment of the ligand-binding domain defined by SEQ ID NO:4 (RMLYFAPDLV), (v) a fragment of the ligand-binding domain defined by SEQ ID NO:5 (FLCMKALLL), and (vi) a fragment of the ligand-binding domain defined by SEQ ID NO:6 (QLTKLLDSV), wherein administration of said vaccine to a subject induces a cytotoxic immune reaction against cells expressing androgen receptor. Other suitable DNA vaccines encode native or modified SSX2 as described in Smith et al. 2011 (Vaccines targeting the cancer-testis antigen SSX-2 elicit HLA-A2 epitopes specific cytolytic T cells. J. Immunother 2011:34:569-80) and Smith et al. 2014 (DNA vaccines encoding altered peptide ligands for SSX2 enhance epitope-specific CD8+T cell immune responses. Vaccine 2014:32:1707-15), the contents of which are incorporated by reference in their entireties. Another suitable vaccine for prostate cancer is a DNA vaccine encoding prostatic acid phosphatase (PAP), which is disclosed in U.S. Pat. No. 7,179,797, the contents of which are incorporated in its entirety.

Androgen receptor genes are known and have been cloned from many species. For example, the human, mouse, rat, dog, chimpanzee, macaque, and lemur androgen receptor cDNA along with amino acid sequences can be found at GenBank Accession Nos. NM 000044 (SEQ ID NO:9), NM 013476 (SEQ ID NO:10), NM 012502 (SEQ ID NO:11), NM 001003053 (SEQ ID NO:12), NM 001009012 (SEQ ID NO:13), U94179 (SEQ ID NO:14), and U94178 (SEQ ID NO:15), respectively, which are incorporated by reference in their entirety.

In some embodiments, the method of eliciting, increasing or enhancing an anti-tumor response comprises administering a TLR3 agonist in combination with a TLR9 agonist and at least one anti-tumor vaccine, preferably a DNA vaccine.

In another embodiment, the disclosure provides a method of eliciting, increasing or enhancing the immune response to cancer, the method comprising administering at least one TLR3 and at least one TLR9 agonist and at least one cancer vaccine directed to a tumor antigen in a subject, wherein the immune response to the tumor antigen is increased relative to a subject treated with the vaccine alone. In a preferred embodiment, the immune response is a cellular immune response, preferably a CD8+ T cell response.

In a further embodiment, a method for increasing the efficacy of a vaccine, particularly a nucleic acid vaccine (e.g., DNA or mRNA vaccine) is provided. The method comprises the steps of administering at least one TLR3 and at least one TLR9 agonist and at least one nucleic acid vaccine to a subject in need thereof, wherein at least one TLR3 and TLR9 agonist is administered in an amount effective to increase the efficacy of the nucleic acid vaccine. In some preferred embodiments, the vaccine is a DNA vaccine. In some aspects, the administration of the at least one TLRe and TLR9 agonist results in an increase in activation of CD8+ T cells in the subject. Suitably, this increased activation of CD8+ T cells is specific to CD8+ T cells and result in an increased immune response.

The at least one TLR3 agonist and TLR9 agonist may be administered in combination with the vaccine. “Combination” refers to the ability to administer the agonist first, followed by the vaccine, to administer the vaccine first followed by the agonist, or the administration at substantially the same time of the agonist and immunotherapeutic agent.

In some embodiments, the TLR agonist is co-administered intradermal administration with the vaccine (e.g the TLR agonist and vaccine are co-administered). In some embodiments, the TLR agonist is formulated with the vaccine (e.g., DNA vaccine) in a single formulation that is co-administered. In other embodiments, the TLR agonist and vaccine are formulated separately, mixed prior to administration and co-administered to the subject in a single formulation.

In some embodiments, the present disclosure provides compositions for eliciting or increasing an anti-tumor antigen or anti-tumor response, the composition comprising at least one TLR3 agonist, at least one TLR9 agonist and at least one vaccine directed to a tumor antigen (e.g., nucleic acid vaccine) as disclosed herein. The compositions may further comprise at least one pharmaceutically acceptable carrier. The term “pharmaceutically acceptable carrier” refers to any carrier, diluent or excipient that is compatible with the other ingredients of the formulation and not deleterious to the recipient.

As the Examples demonstrate, specific TLR agonists can increase vaccine immunogenicity and the anti-tumor response. TLR agonists treatment in combination with a vaccine, results in an increased anti-tumor response. While TLR agonists have been previously used as stand-alone immune activating agents, potential anti-cancer therapies, and as vaccine adjuvants for the purpose of increasing innate immune stimulation, TLR agonists have not been specifically used for the purpose of reducing immune regulatory signals at the time of vaccine-directed T cell activation, as is provided here.

The inventors have found that TLR3 and TLR9 agonists in combination with a vaccine are unexpectedly more effective and have an increased anti-tumor response. TLR3 and TLR9 agonists have beneficial effects on anti-tumor treatment when combined with vaccination, while other TLR do not have this effect.

As further demonstrated in the Examples, TLR stimulation of APCs leads to IL-12 release that mediates reduced PD-1 expression during T-cell activation.

Aspects of the present disclosure that are described with respect to methods can be utilized in the context of the pharmaceutical compositions or kits discussed in this disclosure. Similarly, aspects of the present disclosure that are described with respect to the pharmaceutical compositions can be utilized in the context of the methods and kits, and aspects of the present disclosure that are described with respect to kits can be utilized in the context of the methods and compositions.

This disclosure provides kits. The kits can be suitable for use in the methods described herein. Suitable kits include a kit for treating cancers comprising a composition comprising at least one TLR3 agonist and at least one TLR9 agonist and a vaccine, particularly a nucleic acid vaccine as described herein. In one aspect, the kit provides composition comprising a TLR3 agonist and TLR9 agonist in amounts effective for increasing the cellular immune response to a tumor antigen (e.g. cancer antigen, preferably prostate cancer antigen). In some aspects, the kits provide at least one TLR3 and TLR9 agonist and at least one vaccine. In some aspects, instructions on how to administer the composition and/or TLR agonists and vaccine are provided. In some aspects, the vaccine is preferably a DNA vaccine.

Suitable kits include a kit for eliciting, increasing or improving an immune response against an antigen or immunogen. The kit comprises at least one TLR3 agonist and at least one TLR9 agonist and an vaccine directed against a tumor antigen or immunogen. In some aspects, the kits provide at least one TLR3 agonist and TLR9 agonist and at least one vaccine. In some aspects, instructions on how to administer the composition and/or TLR agonists and vaccine are provided. In some aspects, the vaccine is preferably a DNA vaccine.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim.

The following non-limiting examples are included for purposes of illustration only and are not intended to limit the scope of the range of techniques and protocols in which the compositions and methods of the present invention may find utility, as will be appreciated by one of skill in the art and can be readily implemented.

### EXAMPLES

Using the ova tumor model, the inventors have surprisingly found that stimulation of TLRs (notably using TLR3 or TLR9 agonists in combination) at the time of T cell activation with an tumor antigen, e.g. DNA vaccine encoding a peptide, can affect the magnitude of and duration of CD8+T cell activation. Moreover, delivery of TLR3 agonist or TLR9 agonist with a DNA vaccine elicited greater anti-tumor responses to a PD-L1-expressing syngeneic tumor in vivo. Thus, TLR treatment by specific TLRs (e.g., TLR3 and TLR9 in combination) at the time of T cell activation surprisingly elicits active tumor-lytic CD8+ T cells in the presence of a preexisting immunosuppressive tumor. In general, therefore, the efficacy of vaccines might be greatly augmented by targeting the upregulation or function of specific regulatory molecules at the time of T cell activation by using TLR3 agonist and TLR9 agonist.

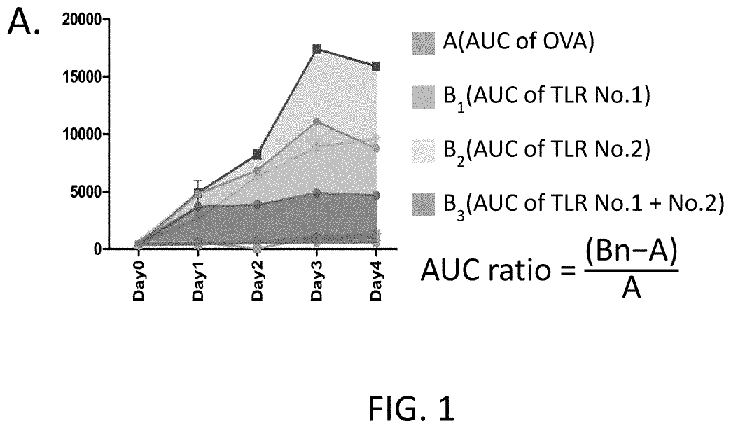

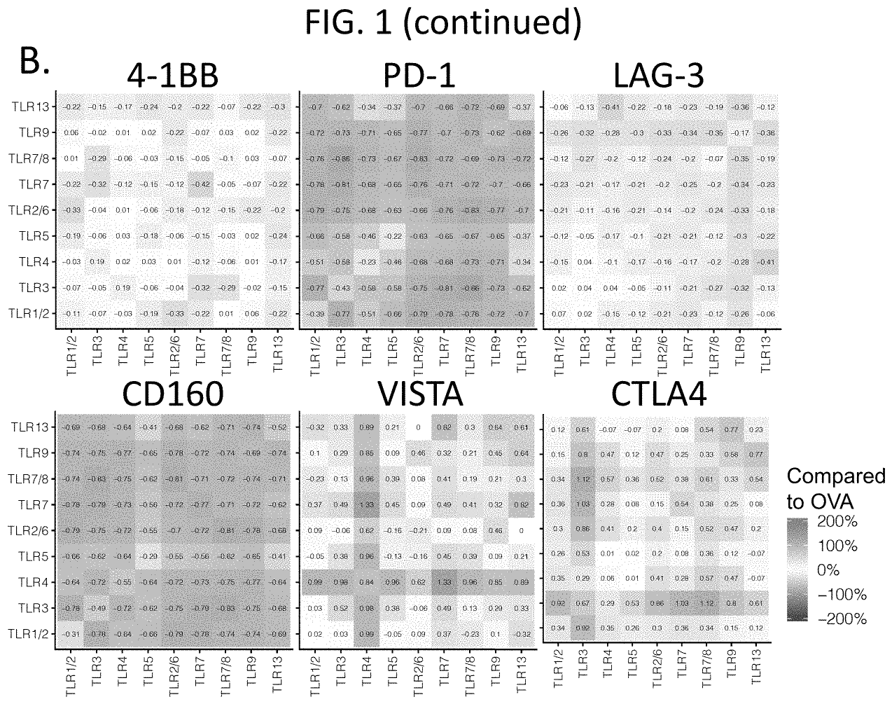

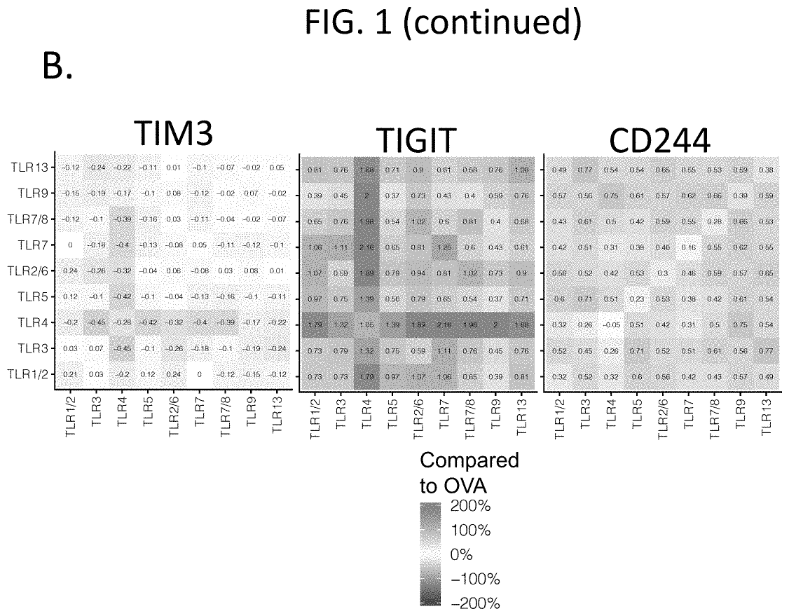

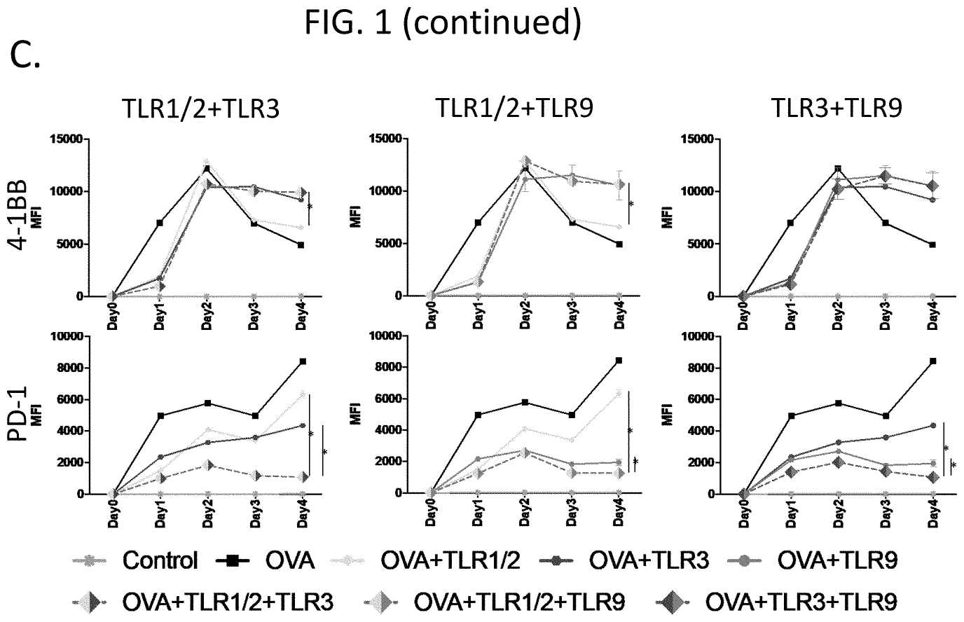

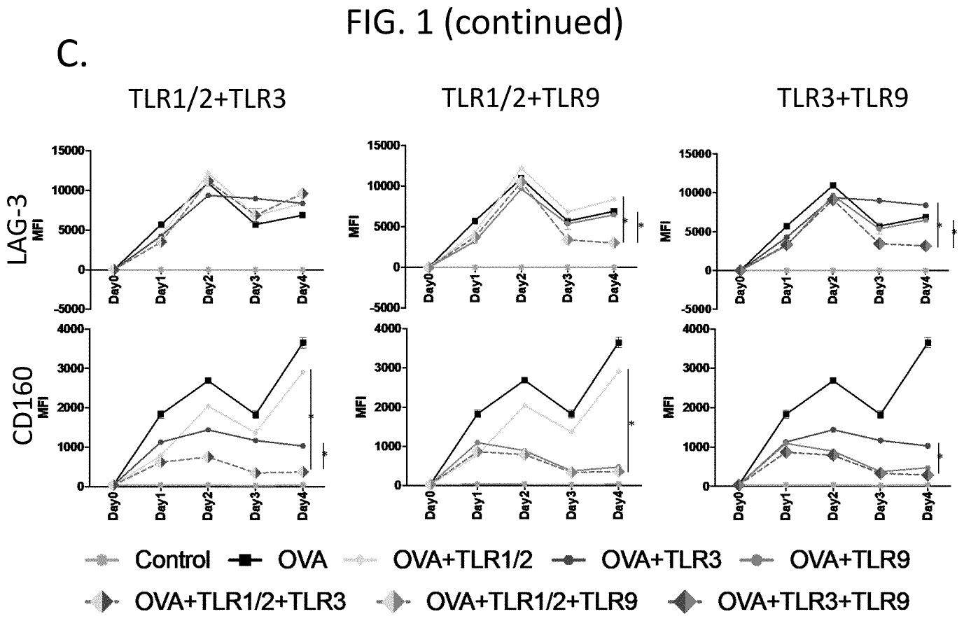

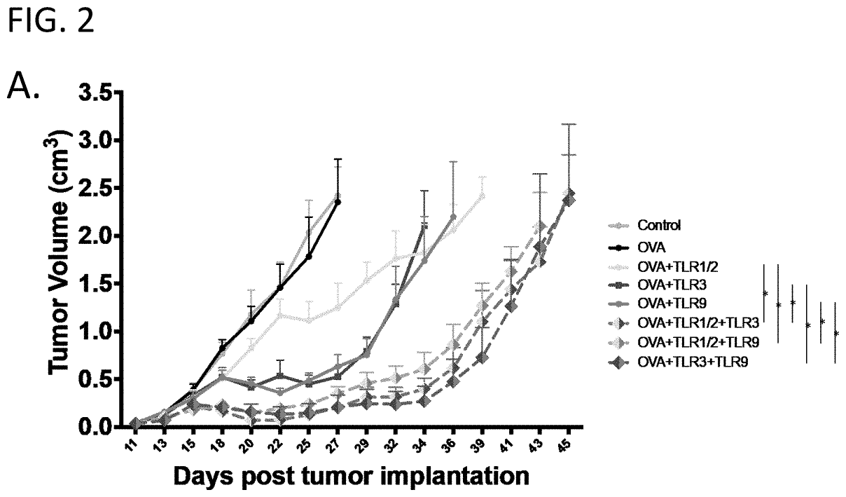

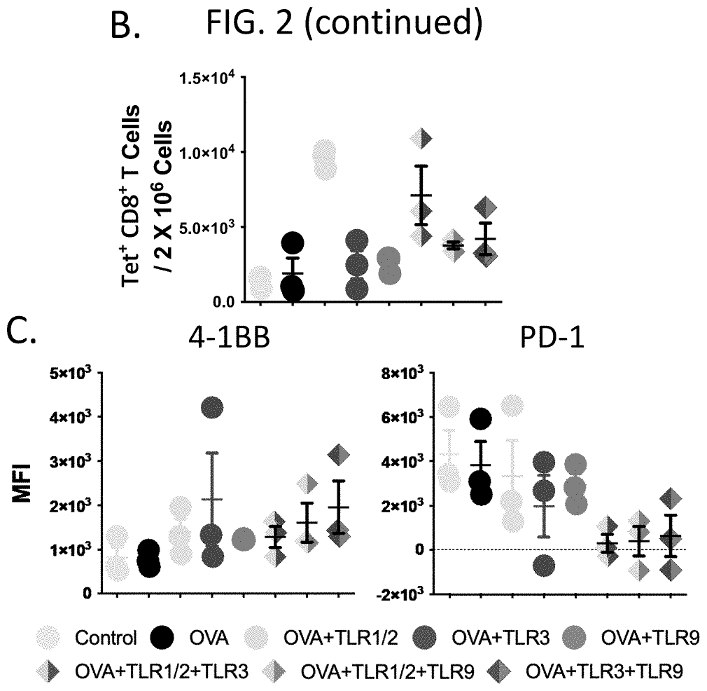

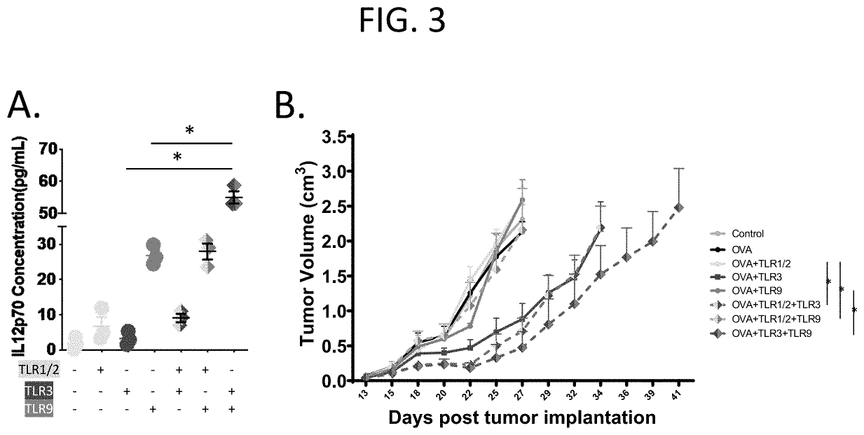

FIG. 1A demonstrates the 4-1BB and T-cell checkpoint receptor expression on CD8+T cells. FIG. 1B shows a heat-map demonstrating AUC ratio of each pairwise combination for 4-1BB and multiple T-cell checkpoint receptors. FIG. 1C shows 4-1BB, PD-1, LAG-3, and CD160 expression with the combinations of TLR1/2, TLR3, and TLR9 agonists. FIG. 2 further demonstrates that the combination of TLR agonists elicits greater antitumor immunity in vivo. FIG. 2A shows tumor growth curves (median+standard error, n=6 to 7 animals per group) and FIG. 2B demonstrates the percentage of infiltrating tetramer⁺CD8⁺ T cells among CD45⁺ cells. Lastly, tumor-infiltrating tetramer⁺CD8⁺ T cells were evaluated for 4-1BB and PD-1 expression by flow cytometry, demonstrating low PD-1 expression in the TLR3 and TLR9 combination treatment. The anti-tumor effects of TLR combinations not strictly dependent on IL12 as demonstrated in FIG. 3.

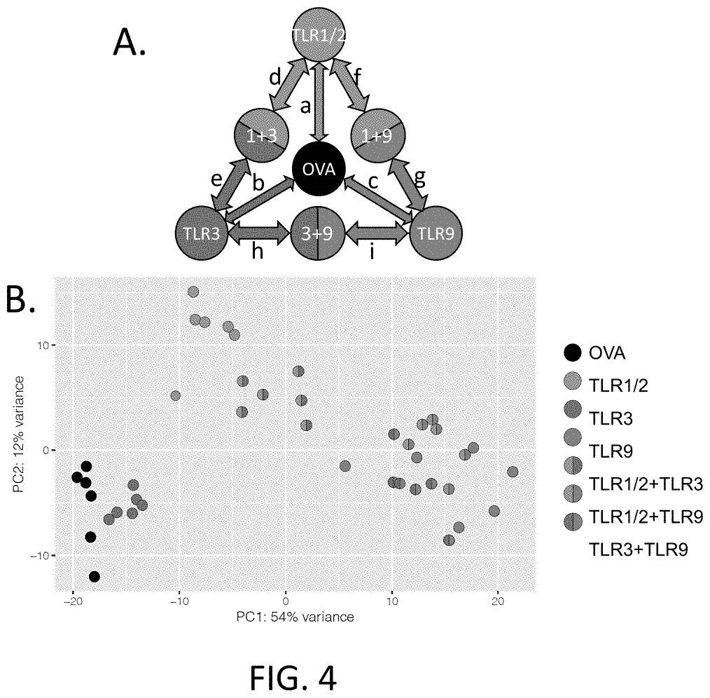

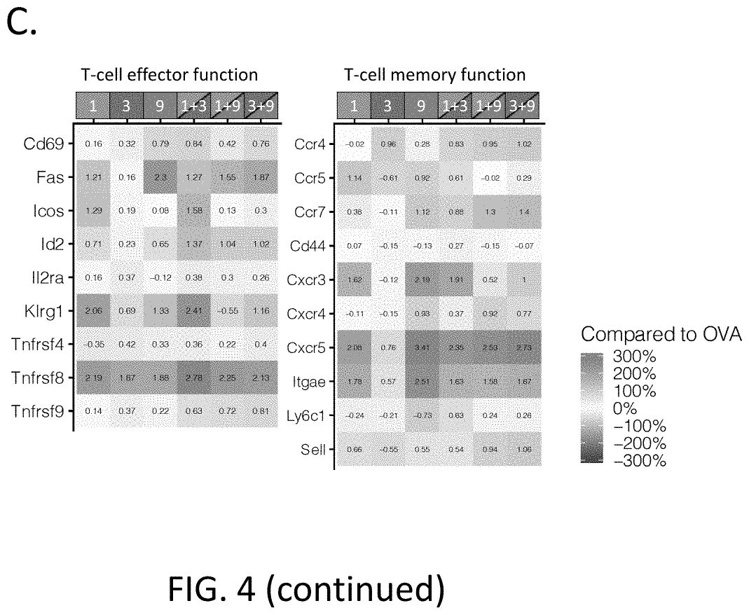

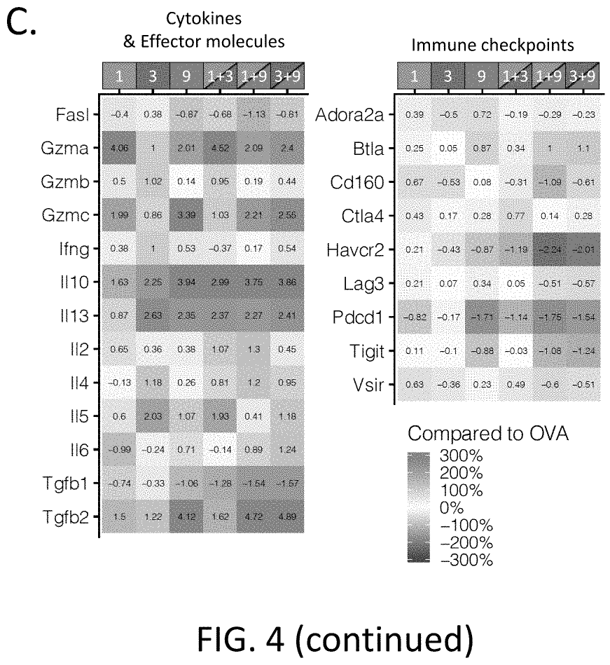

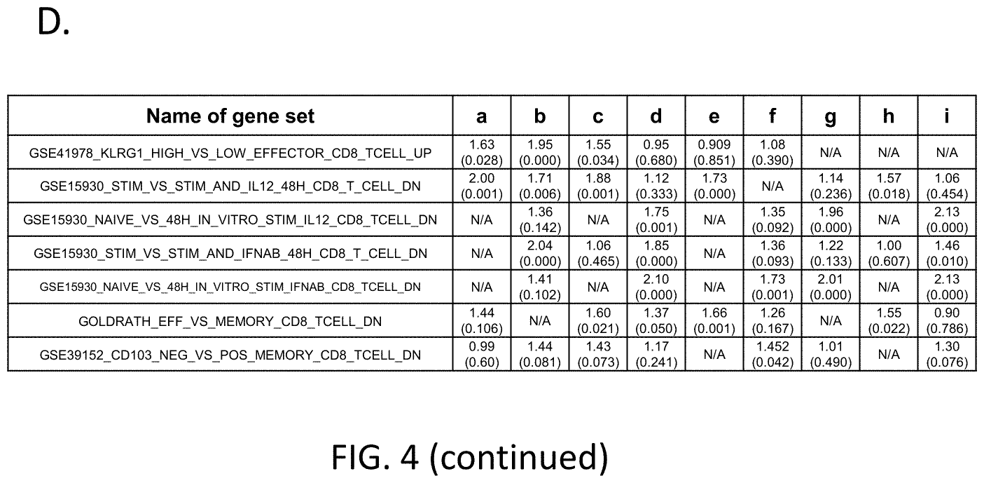

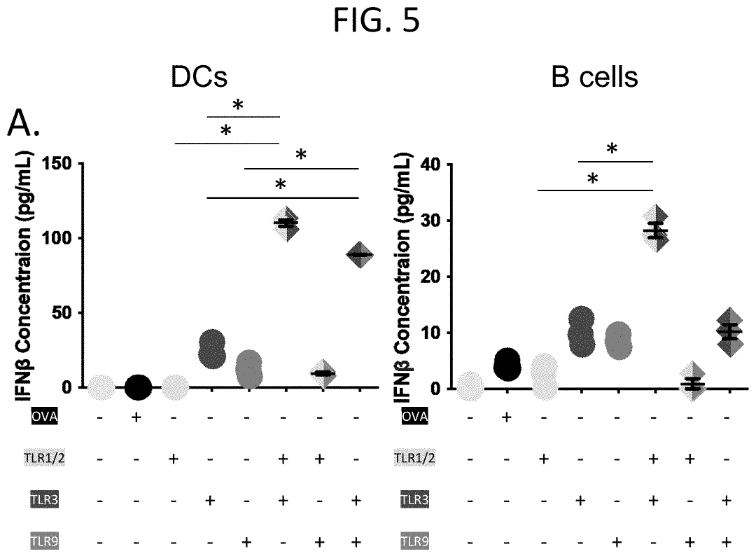

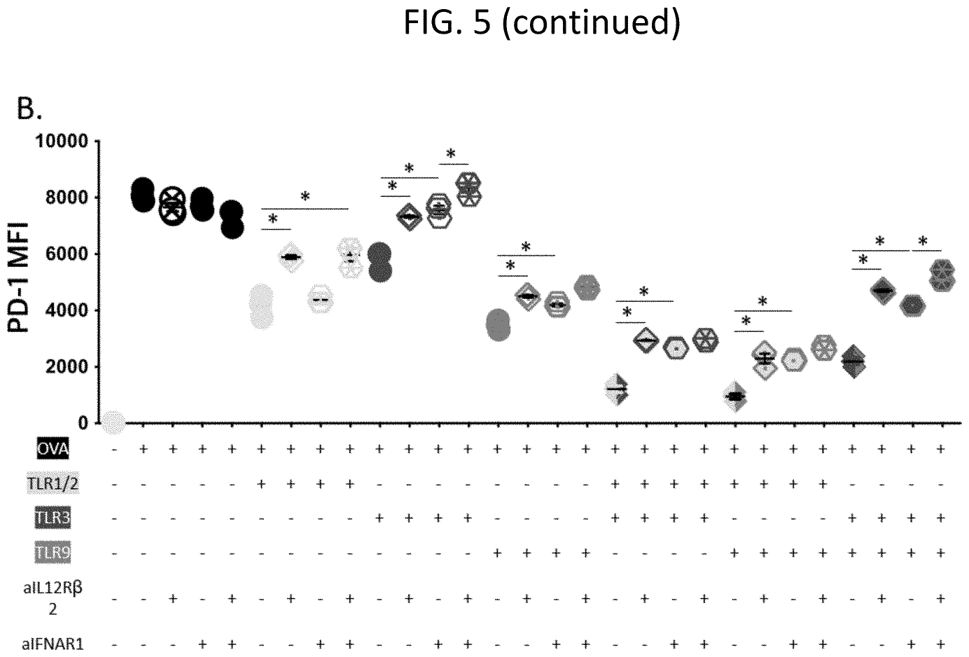

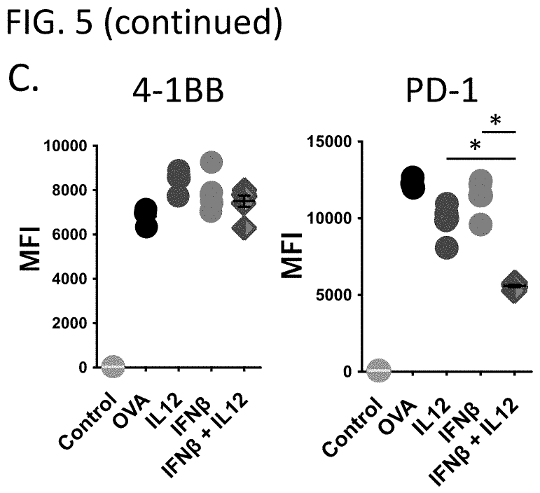

FIG. 4 further demonstrates that the CD8⁺ T cells activated in the presence of TLR3 and TLR9 agonists exhibit distinct gene expression profiles with differences in effector and memory function. We further demonstrate that the changes in PD-1 expression on the CD8+T cells from TLR stimulation depends on both IL-12 and type 1 interferons (FIG. 5).

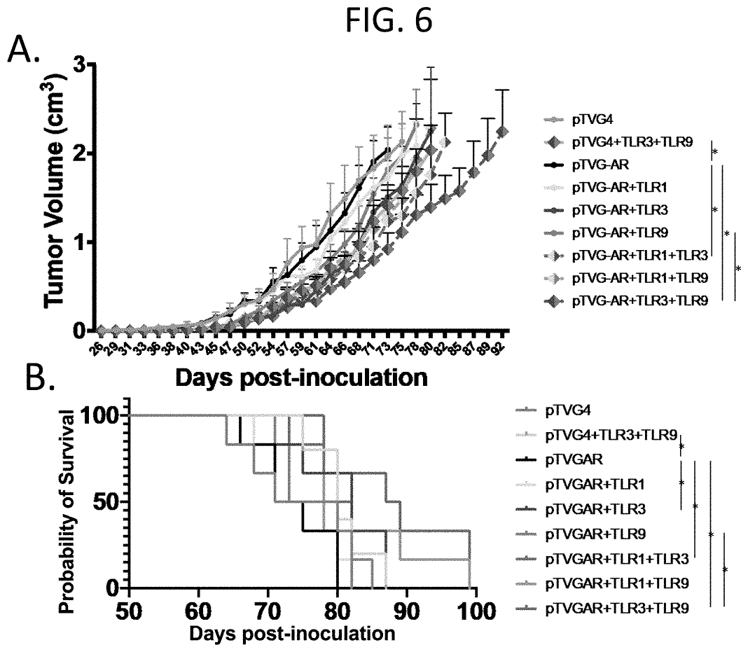

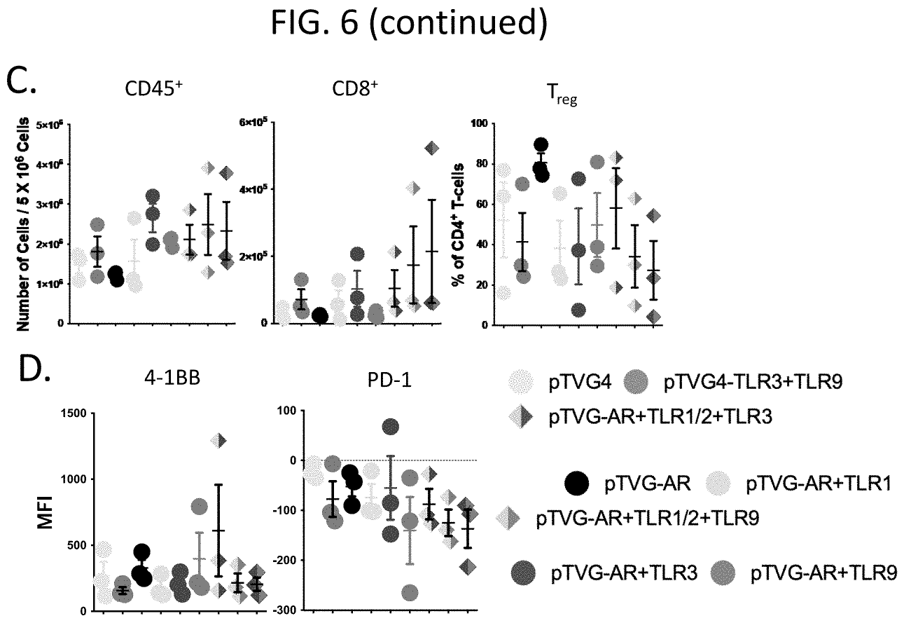

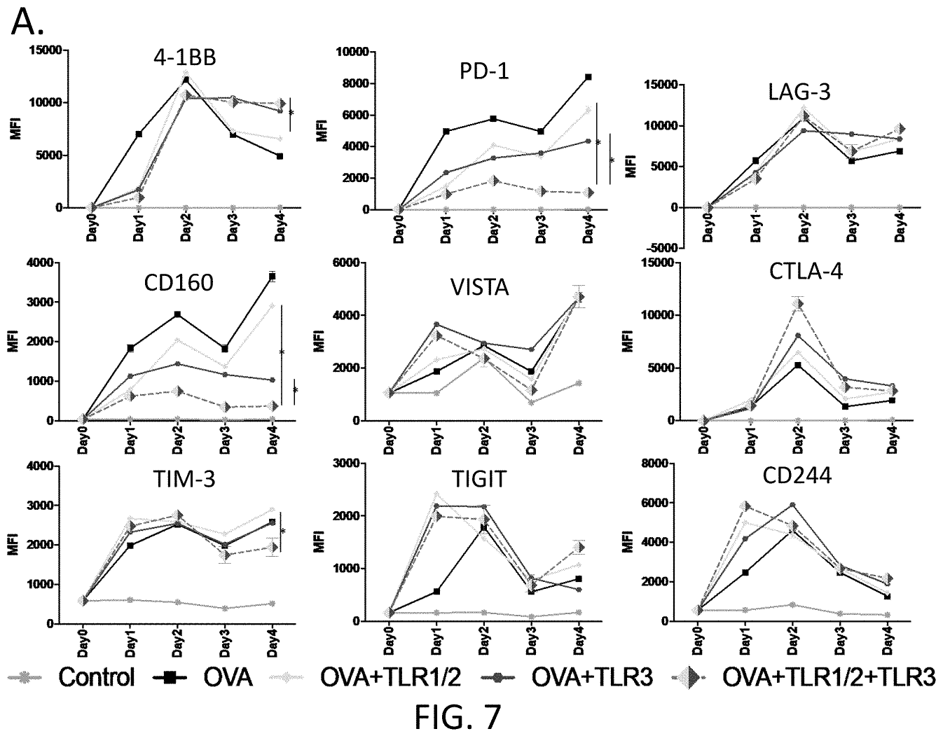

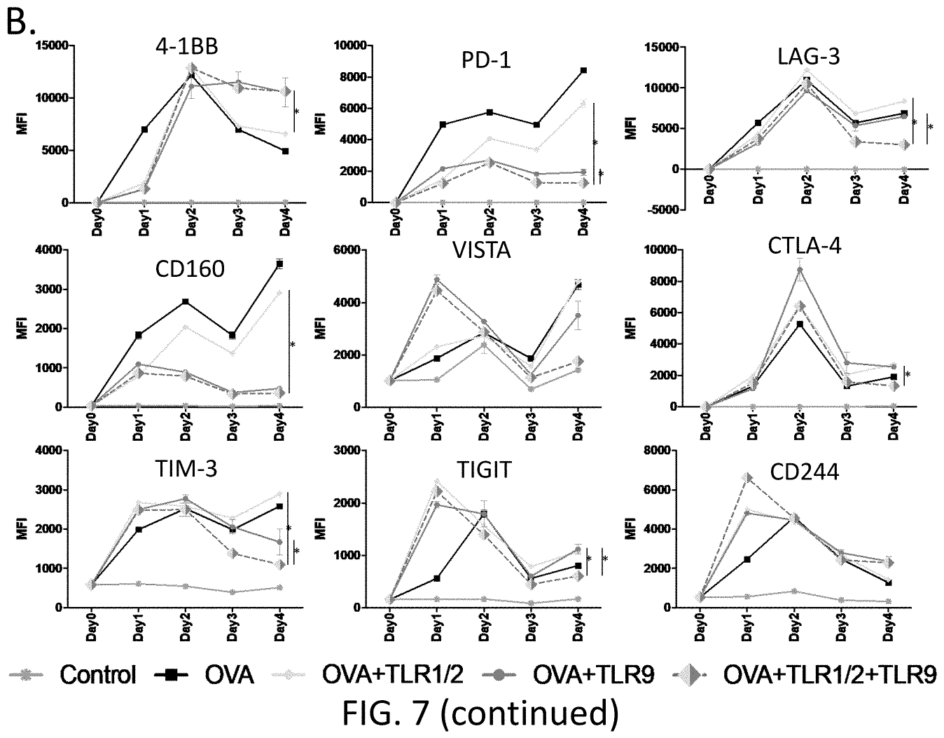

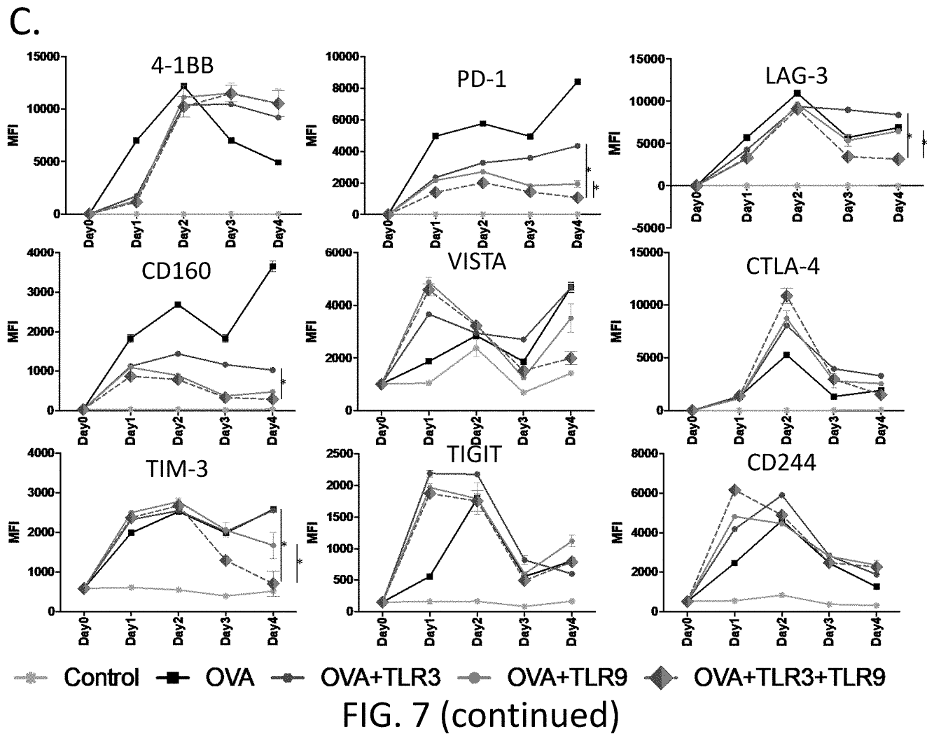

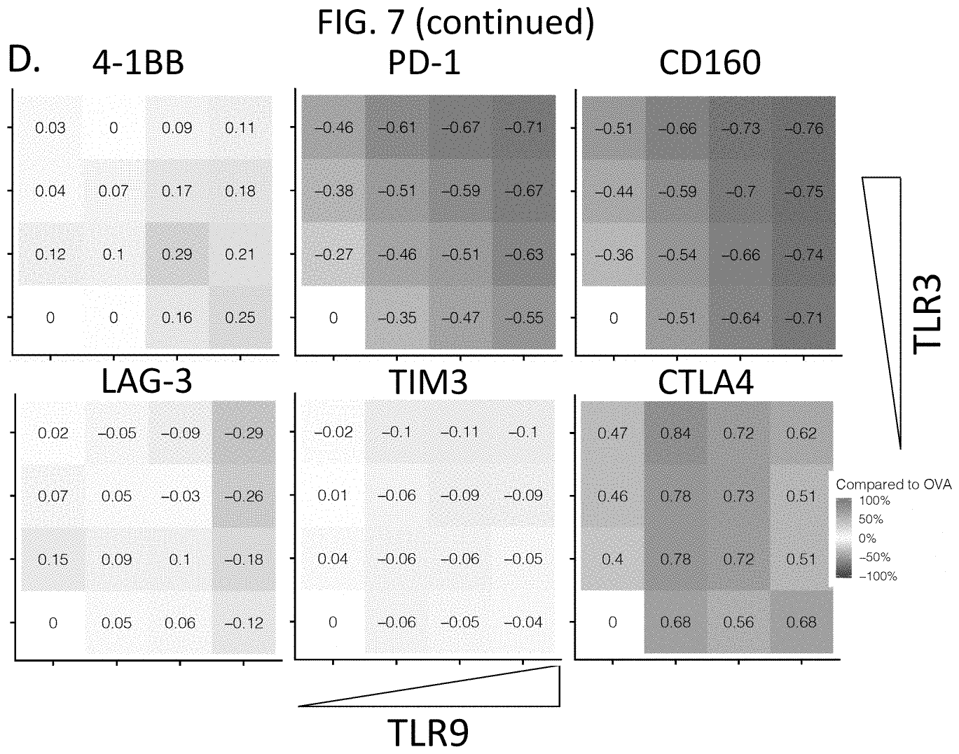

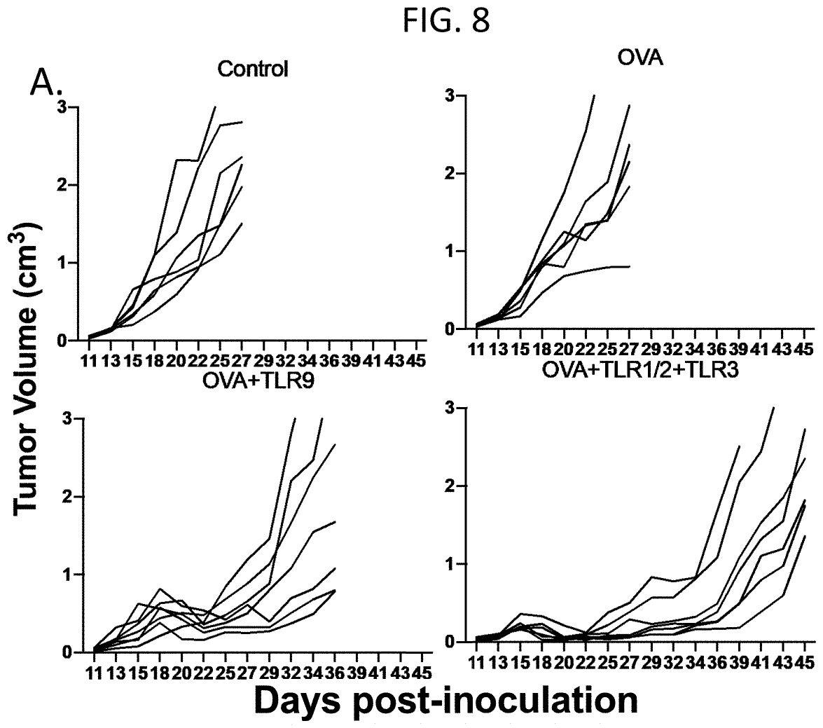

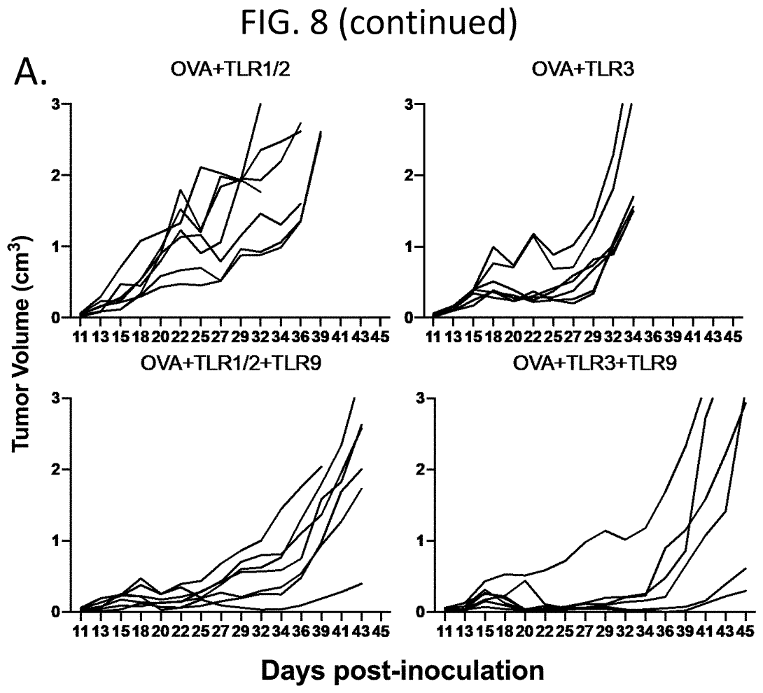

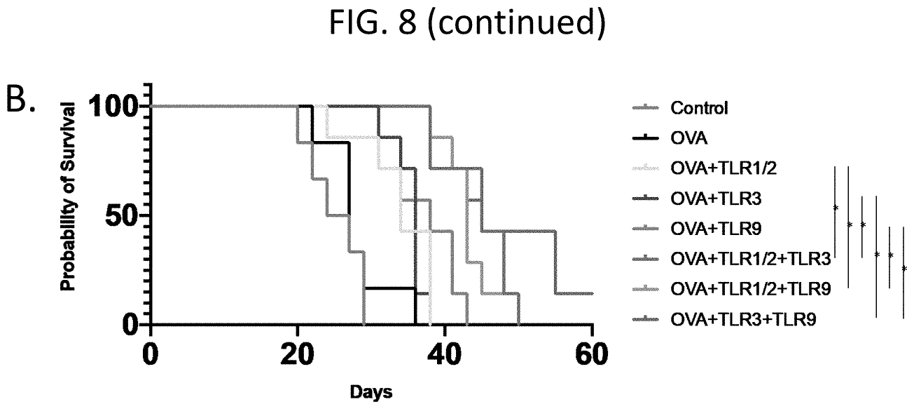

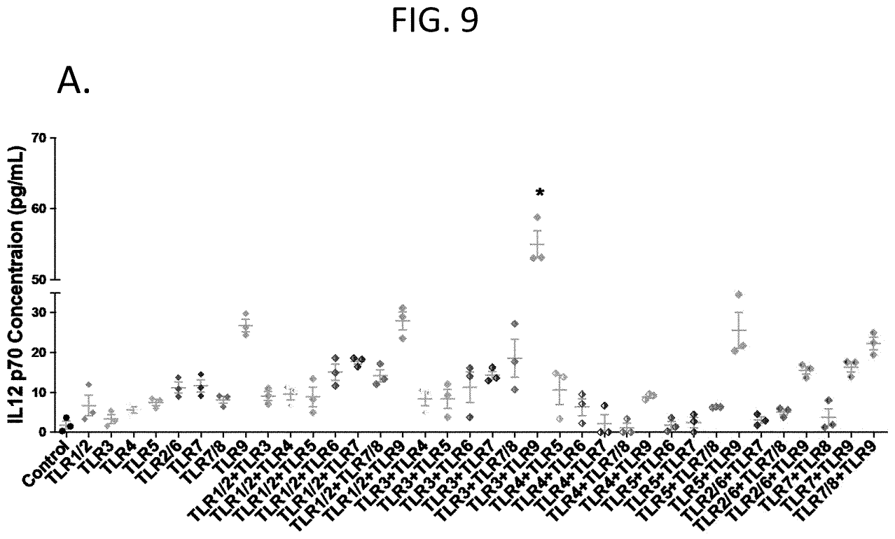

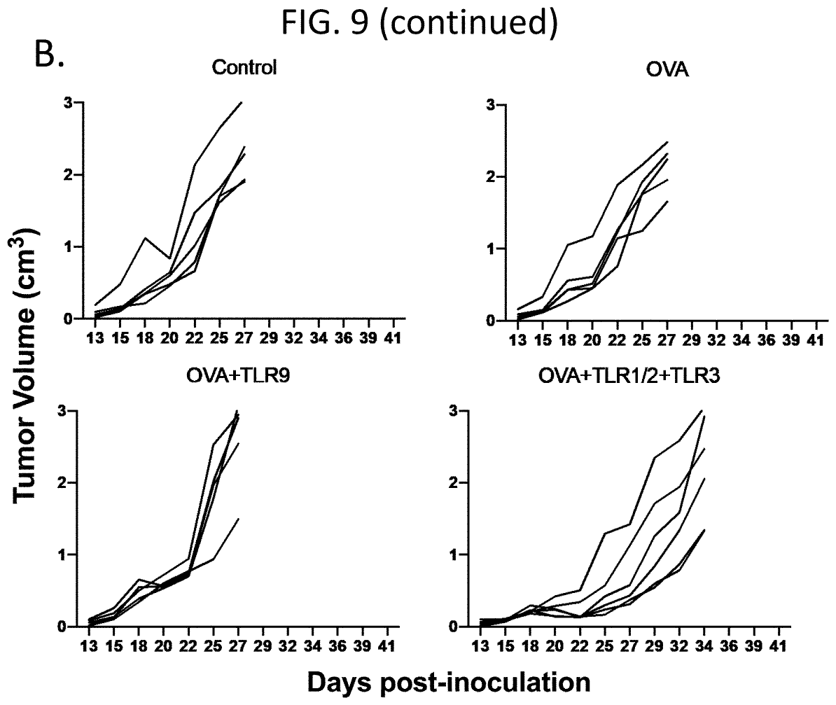

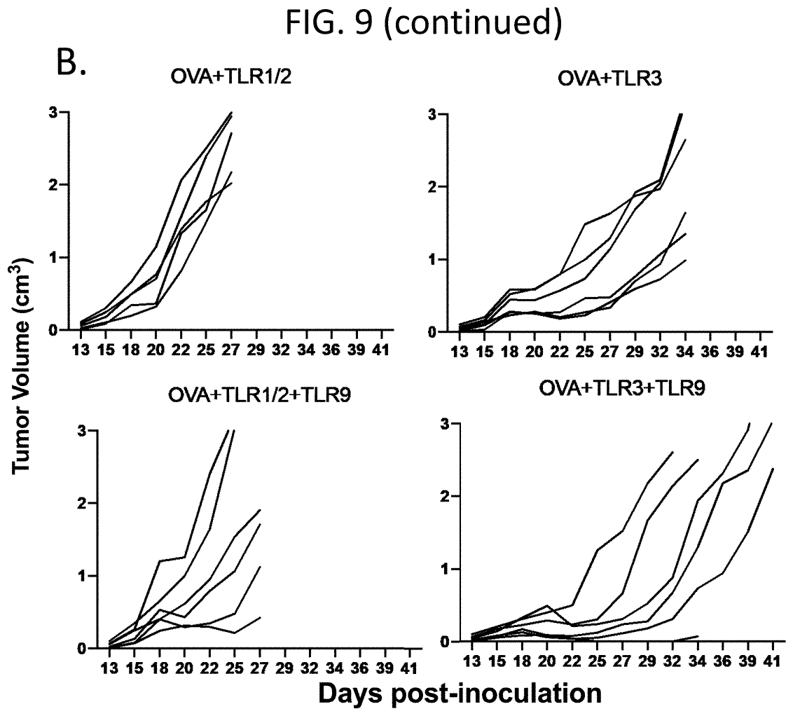

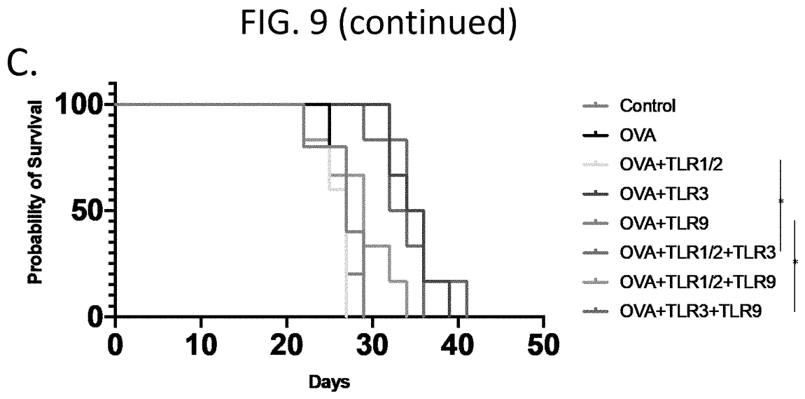

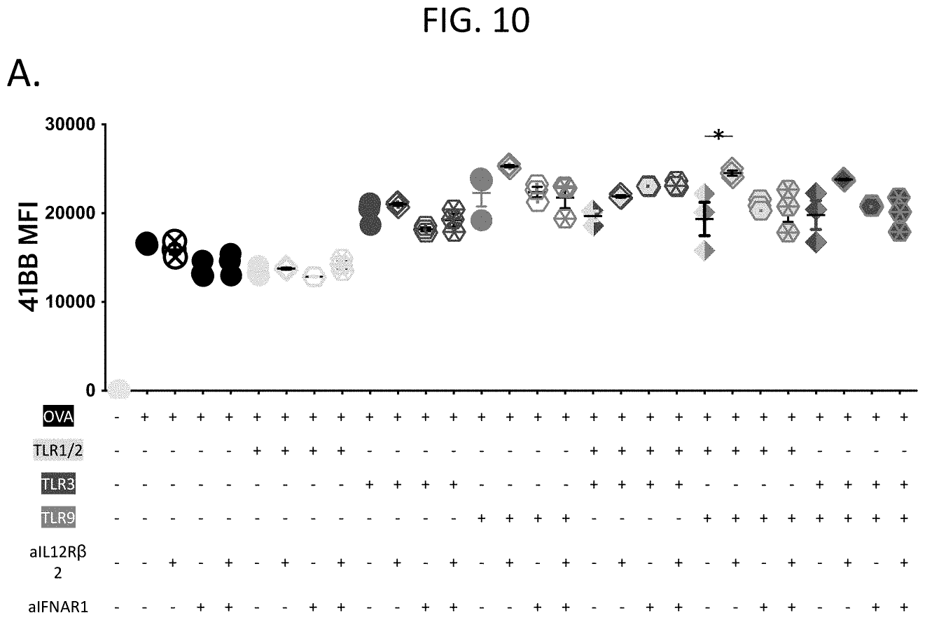

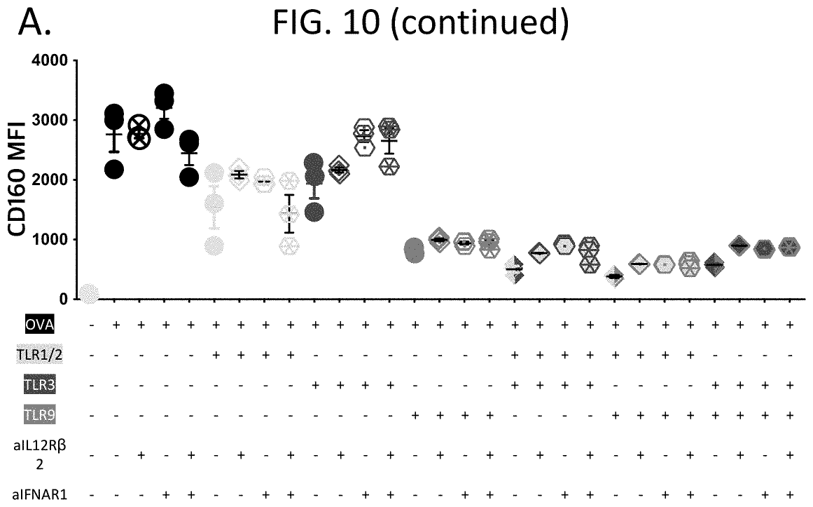

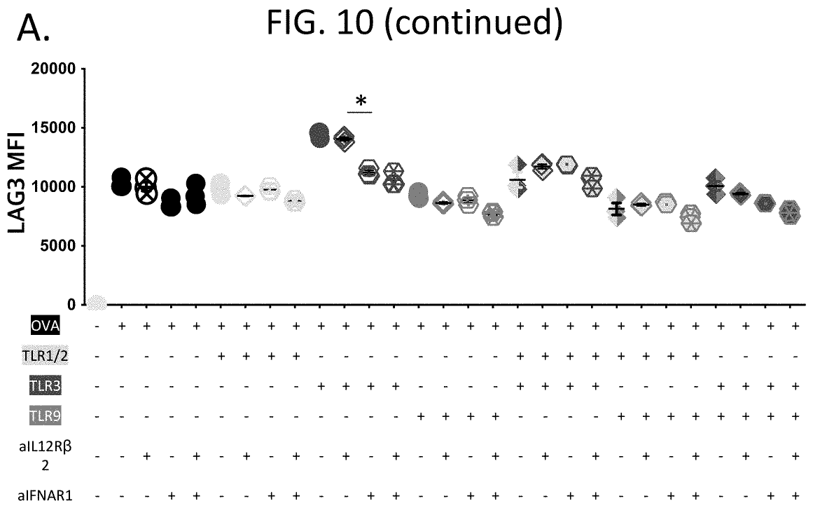

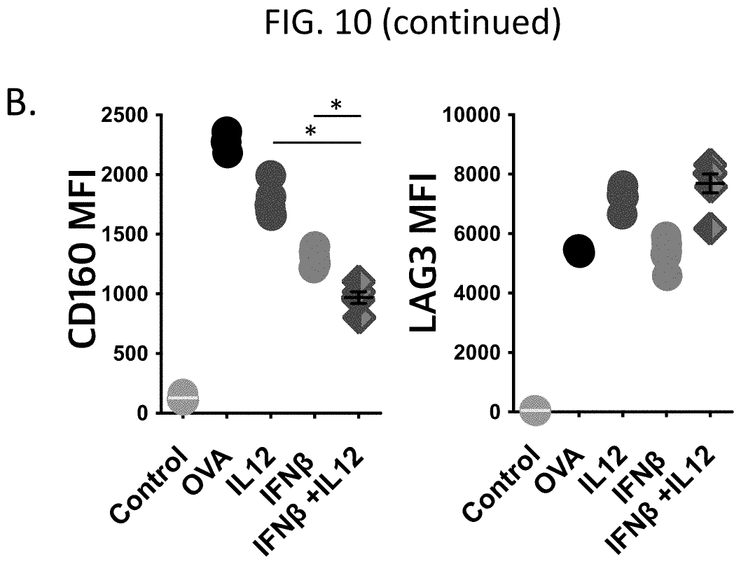

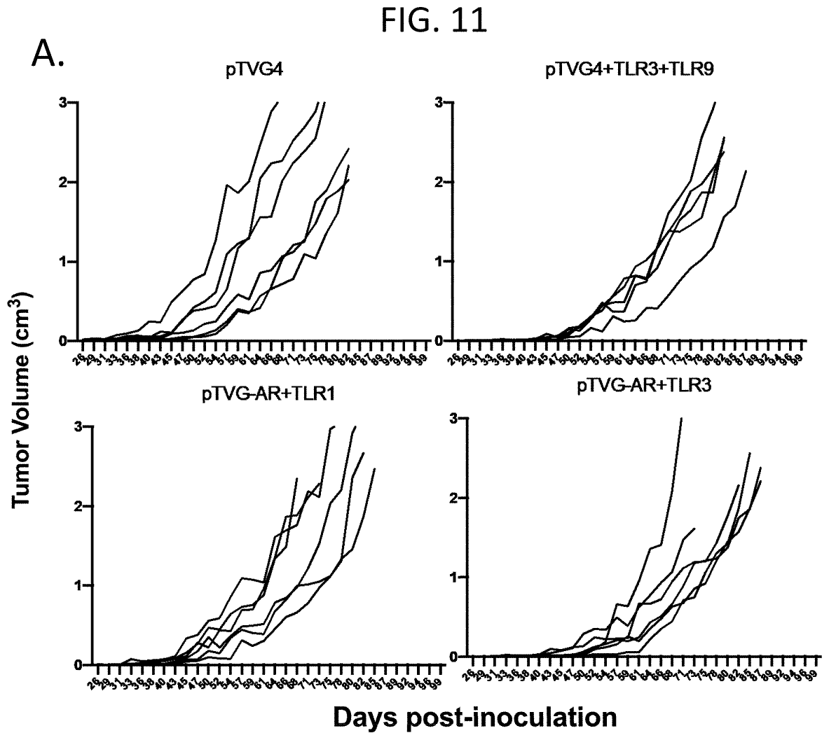

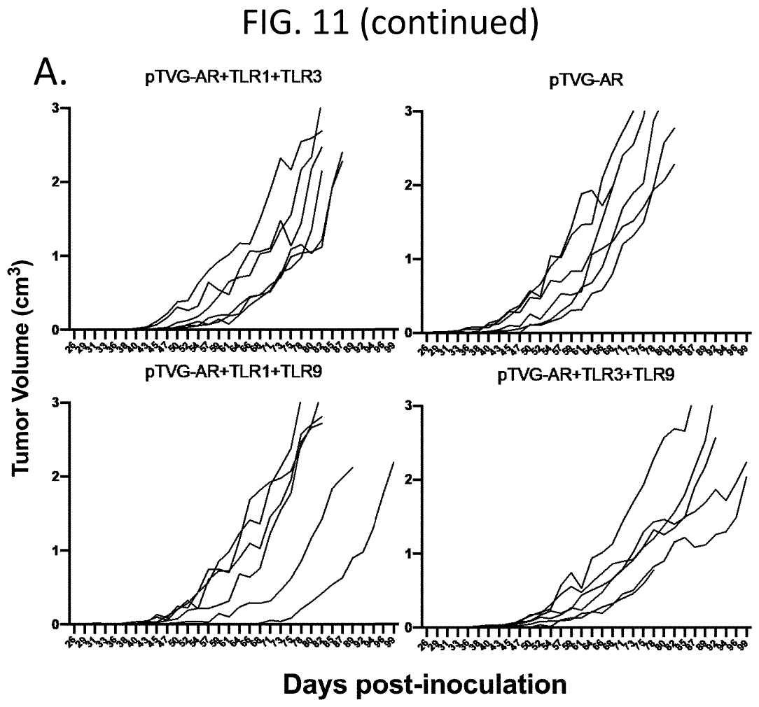

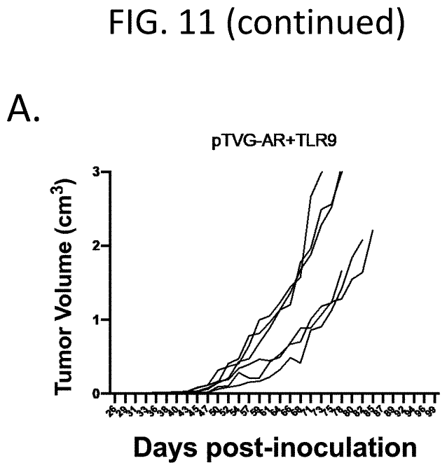

Using a prostate cancer tumor model, the inventors have demonstrated that the combination of TLR agonists TLR3 and TLR9 are able to unexpectedly improve the anti-tumor efficacy of a DNA vaccine (FIG. 6 and FIG. 11). The TLR agonists elicits greater antitumor immunity in vivo (FIG. 8).

Our findings demonstrate that specific TLR3 and TLR9 stimulation in combination can affect the transcriptional profile of CD8+ effector cells, favoring establishment of effector memory cells. Our findings demonstrate that the combination of TLR3 and TLR9 can be used to increase the anti-tumor efficacy of CD8+ effector memory cells with vaccination.

### Materials and Methods

### Mice

OT-1 (Stock No: 003831), C57BL/6 J (Stock No: 000664), and IL-12p40 KO (Stock No: 002693) mice were purchased from The Jackson Laboratory (Jax, Bar Harbor, Mass.). Mice were maintained in microisolator cages under aseptic conditions. All experiments were conducted under an IACUC-approved protocol that conforms to the NIH guide for the care and use of laboratory animals.

### Cell Lines

E.G7-OVA (derivative of EL4) cells were obtained from ATCC (Manassas, Va., Cat. #CRL-2113) and maintained via the ATCC-recommended culture methods. E.G7-OVA cells were lentivirally transduced to express PD-L1, as previously described [1]. The TRAMP-C1 cell line was obtained from ATCC (Cat #CRL-2730) and cultured according to their instructions.

[1] Zahm CD, Colluru VT, McNeel DG. Vaccination with high-affinity epitopes impairs antitumor efficacy by increasing PD-1 expression on CD8(+) T cells. Cancer Immunol Res. 2017; 5(8):630-641. doi:10.1158/2326-6066.CIR-16-0374.

### Peptides

Peptide for the H2Kb-restricted epitope from chicken ovalbumin (SIINFEKL; SEQ ID NO: 1) was synthesized, and the purity and identity were confirmed by mass spectrometry and gas chromatography (LifeTein, LLC., Hillsborough, N.J.). Peptide was reconstituted in DMSO (2 mg/ml) and stored at −80° C. until use.

### In Vitro Assays

### OT-1 Splenocyte Stimulation

Splenocytes were isolated from the spleens of OT-1 mice, disaggregated using a mesh screen, and then treated to osmotically lyse red blood cells with an ammonium chloride/potassium chloride lysis buffer (0.15 M NH₄Cl, 10 mM KHCO₃, 0.1 mM EDTA). Splenocytes were then cultured in RPMI 1640 medium supplemented with L-glutamine, 10% fetal calf serum (FCS), 200 U/mL penicillin/streptomycin, 1% sodium pyruvate, 1% HEPES, and 50 μM β-MeOH with 2 μg/mL SIINFEKL (SEQ ID NO: 1) peptide. TLR agonists were purchased from InvivoGen (San Diego, Calif.) and added 1 hour before stimulating with the peptide at the following concentrations: 300 ng/mL Pam3CSK4, 10 mg/mL Poly(I:C) HMW, 10 mg/mL MPLAs, 100 ng/mL FLA-ST, 100 ng/mL FSL-1, 3 mg/mL Gardiquimod, 10 mg/mL R848, 5 mmol/L ODN 1826, 2 μg/mL ORN Sa19. At the time points indicated, cells were analyzed via flow cytometry with the following antibodies: CD3-FITC(BD 555274), CD4-BUV395 (BD 563790), CD8-BV786(BD 563332), LAG-3-BV711(BD 563179), PD1-PECF594(BD 562523), TIM3-APC(eBioscience 17-5871-82), CTLA4—PECy7 (Tonbo 60-1522-U100), CD160-PE(eBioscience 12-1601-81), TIGIT-BV605 (BD 744212), CD244.2-BV510 (740115), VISTA-BV421 (BD 150212), 41BB-PerCPeF710 (eBioscience 46-1371-82), and Live/Dead Ghost dye 780 (Tonbo, San Diego, Calif. 13-0865—T100). 4-day median fluorescence intensity (MFI) values of immune checkpoint receptors from flow cytometry were computed as Area Under the Curve (AUC). AUC was calculated with the trapezoid rule, using GraphPad Prism (version 9). Each MFI was computed to the AUC ratio compared to OVA stimulation group, with the following equation: (B−A)/A, A: AUC of receptor expression following OVA stimulation without TLR, B: AUC of receptor expression with TLR agonist(s) stimulation. Heat-maps of AUC ratios were generated by R 3.3.1.

### IL12p70 and IFNβ ELISA

Dendritic cells (DCs) were enriched from splenocytes of C57BL/6 mice inoculated with Flt3 ligand-expressing B16 tumor cells using PE-labeled antibodies specific for CD11c (StemCell, Vancouver, Canada, Cat. #17684) as previously described [1]. B cells were enriched from splenocytes of six- to ten-week-old C57BL/6 mice, using a mouse B cell isolation kit (Akadeum, Ann Arbor, Mich., Cat. #12210-110). Cells were stimulated with TLR agonists as above for 24 hours, and culture supernatant was evaluated for the presence of IL12 p70 or IFNβ by ELISA (R&D systems, Minneapolis, Minn., Cat. #DY419-05, DY8234-05), according to the manufacturer's instructions.
[1] Kapadia D, Sadikovic A, Vanloubbeeck Y, Brockstedt D, Fong L. Interplay between CD8alpha+dendritic cells and monocytes in response to Listeria monocytogenes infection attenuates T cell responses. PloS One. 2011; 6(4):e19376. doi:10.1371/journal. pone.0019376.

### RNA Preparation and Sequencing

OT-1 splenocytes were stimulated in vitro as above, and CD8+ T cells were isolated via immunomagnetic negative selection (Stemcell Technologies; 19853). RNA was purified according to the manufacturer's instruction (Direct-zol RNA MiniPrep Plus w/TRI Reagent, Zymo Research), and stored at −80° C. until analysis. Total RNA was submitted to the University of Wisconsin-Madison Biotechnology Center and verified for purity and integrity via the NanoDropOne Spectrophotometer and Agilent 2100 BioAnalyzer, respectively. Samples were then prepared according the TruSeq® Stranded mRNA Sample Preparation Guide (Rev. E) using the Illumina® TruSeq® Stranded mRNA Sample Preparation kit (Illumina Inc., San Diego, Calif.). For each library preparation, mRNA was purified from 1 μg total RNA using poly-T oligo-attached magnetic beads. The mRNA fragments were converted to double-stranded cDNA (ds cDNA) using SuperScript II (Invitrogen, Carlsbad, Calif., USA), RNaseH and DNA Pol I, primed by random primers. The ds cDNA was purified with AMPure XP beads (Agencourt, Beckman Coulter), and products were incubated with Klenow DNA Polymerase to add an ‘A’ base (Adenine) to the 3′ end of the blunt DNA fragments. DNA fragments were ligated to Illumina unique dual adapters, and the adapter-ligated DNA products were purified with AMPure XP beads. Adapter ligated DNA was amplified in a Linker Mediated PCR reaction (LM-PCR) for 10 cycles using Phusion™ DNA Polymerase and Illumina's PE genomic DNA primer set followed by purification with AMPure XP beads. Finally, the quality and quantity of the finished libraries were assessed using an Agilent Tapestation 4200 DNA1000 kit (Agilent Technologies, Inc., Santa Clara, Calif., USA) and Qubit® dsDNA HS Assay Kit (Invitrogen, Carlsbad, Calif., USA), respectively. Libraries were standardized to 2 nM and sequenced on a NovaSeq 6000 (Illumina Inc.). Paired-end, 250 bp sequencing was performed. Data was processed with bcl2fastq (Illumina Inc.).

### RNA-Seq Data Analysis

Data quality was examined by FastQC with per-base sequence quality scores. Data that passed the quality control were aligned to the mouse reference genome using RNA STAR. The expression level of each gene was calculated by FeatureCounts, and the heat-maps for gene of interest were generated using R 3.3.1. Gene expression profiles were subsequently used for differential gene expression analysis using DESeq2. The false discovery rate was controlled using the Benjamini-Hochberg procedure. Rank lists for Gene Set Enrichment Analysis (GSEA) were generated from DESeq2 results, with the following formula: ‘Sign(log 2FoldChange) X −log 10(p-value)’. Pre-ranked GSEA was performed with the Molecular Signatures Database (MSigDB) immunologic signature gene sets.

### Tumor Treatment Studies

Tumor Studies with e.g 7-OVA Tumors

Ovalbumin-expressing E.G7 (PD-L1high) cells were injected subcutaneously into 6-week old female C57BL/6 mice or 6- to 7-week-old female IL12p40 KO mice. When tumors were palpable and similarly sized (0.1 cm3), 2×106 naive OT-1 splenocytes were adoptively transferred to each mouse intraperitoneally. The following day, mice were immunized subcutaneously with 100 μg SIINFEKL peptide with or without TLR agonist(s) as described. Tumor volume was measured using calipers and calculated in cubic centimeters according to the formula: (p/6)×(long axis)×(short axis)2. Obtained tumors were digested in collagenase and DNAse I for 1 hour at 37° C., passed through a 100-mm mesh screen, and analyzed by flow cytometry.

Tumor Study with TRAMP-C1 Tumors

Six- to ten-week-old male C57BL/6 mice were inoculated with 1×106 TRAMP-C1 cells, administered subcutaneously with matrigel. Each mouse was then immunized intradermally with 100 μg DNA vaccine (pTVG-AR) (or vector control) weekly, beginning 1 day after tumor implantation. TLR agonist(s) were co-administered with the vaccine intradermally, and tumor volumes were measured as described above. Obtained tumors were digested in collagenase and DNAse I for 1 hour at 37° C., passed through a 100-mm screen, and analyzed by flow cytometry.

### Statistical Analysis

Group mean comparisons were performed using GraphPad Prism software, v8.4.3. Analysis of Variance (ANOVA) followed by the Tukey's multiple-comparison test was used to compare individual group means. For samples for which ANOVA was not applicable, the linear mixed effects model with Geisser-Greenhouse correction was used to compare group means among treatment groups. Survival analysis was conducted using a Mantel-Cox log-rank test. For all comparisons, P values equal to or less than 0.05 were considered statistically significant.

We claim:

1. A method of eliciting an anti-tumor response in a subject in need thereof, the method comprising administering at least one TLR3 agonist, at least one TLR9 agonist, and an anti-tumor vaccine to the subject in an effective amount to elicit an anti-tumor response in the subject.

2. The method of claim 1, wherein the anti-tumor response is a cell-mediated response.

3. The method of claim 1, wherein the anti-tumor response is a CD8+ T cell response.

4. The method of claim 1, wherein the method comprises administering a TLR3 agonist in combination with a TLR9 agonist prior to administering the anti-tumor vaccine.

5. The method of claim 1, wherein the anti-tumor vaccine is a DNA vaccine.

6. The method of claim 5, wherein the DNA vaccine is specific for prostate cancer.

7. The method of claim 1, wherein the anti-tumor response is to a prostate tumor.

8. The method of claim 1, wherein the subject has prostate cancer.

9. The method of claim 1, wherein the anti-tumor vaccine is a recombinant DNA vaccine comprising a polynucleotide operably linked to a transcriptional regulatory element wherein the polynucleotide encodes an androgen receptor or a fragment of the androgen receptor selected from the group consisting of (i) a mammalian androgen receptor, (ii) a fragment of the androgen receptor that comprises a ligand-binding domain, (iii) a fragment of the ligand-binding domain defined by SEQ ID NO:9, (iv) a fragment of the ligand-binding domain defined by SEQ ID NO:10, (v) a fragment of the ligand-binding domain defined by SEQ ID NO:11, and (vi) a fragment of the ligand-binding domain defined by SEQ ID NO:12.

10. The method of claim 1, wherein the TLR3 agonist is HMW PolyI:C.

11. The method of claim 1, wherein the TLR9 agonist is selected from the group consisting of ODN2006, ODN2216, ODN2395, EMD 1201081, lefitolimod, PF-3512676 (aka CPG7909), ISS 1018, IMO-2055, CpG-28, SD-101, CMP-001, IMO-2125 (tilsotolimod), MGN1703, and AZD1419.

12. The method of claim 1, wherein the anti-tumor response to the tumor antigen is increased relative to a subject treated with the vaccine alone.

13. The method of claim 1, wherein the anti-tumor vaccine comprises at least one tumor antigen and the anti-tumor response is directed to the at least one tumor antigen.

14. The method of claim 13, wherein the tumor antigen is a prostate cancer antigen.

15. A kit for eliciting an anti-tumor response, the kit comprising:
at least one TLR3 agonist, at least one TLR9 agonist; and
at least one cancer vaccine.

16. A composition for eliciting an anti-tumor response, the composition comprising at least one TLR3 agonist, at least one TLR9 agonist and at least one anti-tumor vaccine.

17. The composition of claim 16, wherein the composition further comprises a pharmaceutically acceptable carrier.

18. The composition of claim 16, wherein the anti-tumor vaccine comprises a prostate cancer vaccine.

19. The composition of claim 16, wherein the anti-tumor vaccine is a DNA vaccine.

20. The composition of claim 16, wherein the vaccine is a recombinant DNA vaccine comprising a polynucleotide operably linked to a transcriptional regulatory element wherein the polynucleotide encodes an androgen receptor or a fragment of the androgen receptor selected from the group consisting of (i) a mammalian androgen receptor, (ii) a fragment of the androgen receptor that comprises a ligand-binding domain, (iii) a fragment of the ligand-binding domain defined by SEQ ID NO:9, (iv) a fragment of the ligand-binding domain defined by SEQ ID NO:10, (v) a fragment of the ligand-binding domain defined by SEQ ID NO:11, and (vi) a fragment of the ligand-binding domain defined by SEQ ID NO:12, and/or the TLR3 agonist is HMW PolyI:C and the TLR9 agonist is selected from the group consisting of ODN2006, ODN2216, ODN2395, EMD 1201081, lefitolimod, PF-3512676 (aka CPG7909), ISS 1018, IMO-2055, CpG-28, SD-101, CMP-001, IMO-2125 (tilsotolimod), MGN1703, and AZD1419.
